(19) United States
(12) Patent Application Publication
(10) Pub. No.: US 2013/0035271 A1
(43) Pub. Date: Feb. 7, 2013

(54) ESTERS FROM SOLID POLYOLS AND UNSATURATED CARBOXYLIC ACIDS

(71) Applicants: Peter Daute, Beverstedt (DE); Wilhelm Reiners, Loxstedt (DE); Martin Schäfer, Stubben (DE); Udo Frerichs, Loxstedt (DE); Hinrich Hildebrandt, Armstorf (DE); Joern Ellerbrake, Bremerhaven (DE)

(72) Inventors: Peter Daute, Beverstedt (DE); Wilhelm Reiners, Loxstedt (DE); Martin Schäfer, Stubben (DE); Udo Frerichs, Loxstedt (DE); Hinrich Hildebrandt, Armstorf (DE); Joern Ellerbrake, Bremerhaven (DE)

(21) Appl. No.: 13/519,987

(22) PCT Filed: Dec. 30, 2010
(86) PCT No.: PCT/EP2010/007978

§ 371 Date: Oct. 22, 2012

(30) Foreign Application Priority Data
Dec. 30, 2009 (DE) 10 2009 060 865.6

(51) Int. Cl. C07C 67/08; C09K 3/22; C09K 8/32; C09K 3/00; C07C 69/602; C10M 105/38

(52) U.S. Cl. 508/485; 560/198; 507/138; 516/133; 252/88.1

(57) ABSTRACT

The present invention relates to a process for the preparation of an ester from a polyol which is solid at 25° C. and a carboxylic acid component which contains at least 50 wt. % of at least one mono- or polyunsaturated aliphatic carboxylic acid, based on the total weight of the carboxylic acid component, in a reactor under reduced pressure. The invention also provides a device, a process for the preparation of a thermoplastic composition comprising the ester prepared according to the invention, a process for the production of a shaped article comprising the ester according to the invention or the thermoplastic composition according to the invention, a process for the production of a packed product, a process for the production of an at least partly coated object, and uses of the esters according to the invention as an additive in various compositions.

The present invention relates to a process for the preparation of an ester from a polyol which is solid at 25° C. and a carboxylic acid component which contains at least 50 wt. % of at least one mono- or polyunsaturated, aliphatic carboxylic acid, based on the total weight of the carboxylic acid component, in a reactor under reduced pressure. The invention also provides a device, a process for the preparation of a thermoplastic composition comprising the ester prepared according to the invention, a process for the production of a shaped article comprising the ester according to the invention or the thermoplastic composition according to the invention, a process for the production of a packed product, a process for the production of an at least partly coated object, and uses of the esters according to the invention as an additive in various compositions.

Esters, in particular those based on aliphatic carboxylic acids and alcohols, are employed successfully in a large number of uses. In the awareness that raw materials from fossil deposits are becoming scarcer, new sources of raw materials are being sought. Oils from animal or plant renewable raw materials which are broken down to fatty acids e.g. by ozonolysis and refunctionalized or derivatized in further steps appear to be particularly promising.

Ester preparation is an industrially important derivatization, for which various processes are known. These can be classified in various ways. One possibility is classification into low temperature and high temperature processes. In this context, generally, low temperature processes are often more gentle, i.e. generate fewer side reactions and decomposition or oxidation products, and high temperature processes are characterized by higher rates of reaction.

In conventional low temperature processes, as a general rule proton acids or sulphonic acid derivatives are added as catalysts. In the case of proton acids in particular, such as sulphuric acid or phosphoric acid, by-products, in particular unsaturated compounds, are formed in a considerable proportion. The unsaturated substances formed by this procedure are as a general rule coloured, or form coloured compounds with atmospheric oxygen in a short time. This is perceived as a reduction in the product quality of the ester prepared. The unsaturated contents moreover often have the effect of a deterioration in the stability and durability of products to which these esters are added as an additive. The aggressiveness of the acid catalysts at elevated temperature additionally is a burden on the production plants. A usually low rate of reaction is considered to be a further disadvantage of the low temperature processes.

In the high temperature processes, organometallic complexes of the transition metals Ti, Zr, Al, Sn are conventionally employed as catalysts. Because of the high reaction temperature, however, still more coloured by-products are formed, so that expensive working up and/or purification processes become necessary. Furthermore, the removal of the catalyst from the end product is expensive.

EP 0 342 357 A2 describes a device and a process for carrying out esterifications. In this, esters are prepared from alcohols and fatty acids in a production plant at 200 to 250° C., the reaction mixture being led continuously over a particularly hot reaction zone with a short contact time and the preparation being carried out over reaction times of up to 20 hours.

With respect to industrial esterification reactions, there is need for improvement in various aspects in order to meet the requirements and demands of the market. The known processes have at least one, as a rule several of the disadvantages outlined below:
coloured nature or inadequate colourlessness of the products
undesirable by-products,
inadequate stability
low efficiency, high energy consumption, high production costs,
low yield,
impurities, in particular traces of heavy metals,
long reaction times.

There is therefore the need for improvement in the known processes and possibly the provision of new processes in order to be able to provide esters in an improved quality.

There is furthermore the demand for more efficient production processes or devices which have a lower consumption of energy and resources with high conversions, yields and selectivities and render after-treatment steps superfluous.

Furthermore, in particular, suspended substances and the formation of deposits e.g. in hydraulic systems, in particular during long service lives of these hydraulic liquids, are to be reduced. This would prolong the life of seals which are exposed to mechanical or hydraulic stresses and which additionally are subjected to abrasive wear by the suspended substances and deposits described above, in addition to the mechanical stress, which is in any case severe.

Of the large number of industrially available esters, there is interest in partial esters, in particular in those based on glycerol, pentaerythritol, oligomers thereof, and 1,1,1-trimethylolethane, -propane, -butane and -pentane because of their both hydrophilic and hydrophobic properties. A challenge here is to free the products from unreacted alcohol. Furthermore, esters, and in particular partial esters which are based on unsaturated carboxylic acids, are sensitive to oxidation to an increased extent. Such an oxidation is undesirable in the partial ester both as a product and as an additive in compositions and processing products, for example lubricants, since the compositions changed in this way regularly suffer a change in their properties.

The present invention was based on the object of at least partly overcoming the disadvantages emerging from the prior art.

In particular, esters which, as an additive in compositions, contribute as little as possible towards the formation of suspended substances or deposits, or of both, in these compositions were to be provided.

The present invention was also based on the object of providing a process and a device with the aid of which by-products which differ from esters and play a part in the increase in the colour shading of the esters can be reduced, and in this way expensive and time-consuming purification steps can be reduced or even avoided.

A further object of the present invention was to provide additives for the preparation of thermoplastic compositions which, in addition to being environment-friendly, are suitable for modifying the properties of the thermoplastic composition in the desired manner and at the same time for obtaining thermoplastic compositions which meet high requirements, such as in the foodstuffs industry or in medicine.

A contribution towards achieving at least one of the abovementioned objects is made by the subject matter of the category-forming claims, the sub-claims dependent upon these representing further embodiments according to the invention.

A further contribution towards achieving the abovementioned object is made by purification processes or the removal of constituents which tend to precipitate out, or by both.

The present invention provides a process for the preparation of an ester at least based on
a. at least one alcohol component,
b. at least one carboxylic acid component,
c. optionally further additives, and
d. at least one catalyst
   as process components, comprising, in a reactor, the process steps:
i. provision of the process components,
ii. reaction of the process components to give an ester A,
iii. optionally after-treatment of the ester A,
   wherein
the alcohol component comprises at least one polyol which is solid at 25° C., and
the carboxylic acid component comprises at least 50 wt. %, preferably at least 65 wt. %, at least 75 wt. %, or at least 80 wt. % of at least one mono- or polyunsaturated, aliphatic carboxylic acid, based on the total weight of the carboxylic acid components,
   wherein a pressure in a range of 2-600 mbar is applied to the reactor at least during a part of the reaction.

According to a preferred embodiment, such a pressure in a range of from 2 to 100 mbar, particularly preferably 2 to 50 mbar and most preferably 2 to 20 mbar is applied.

According to a further preferred embodiment, the reaction is carried out at a temperature in a range of from 150 to 250° C. Further ranges according to the invention are: from 180 to 230° C., in particular from 200 to 220° C.

In principle, any alcohol component with one or more hydroxyl groups which is known to the person skilled in the art and appears to be suitable for carrying out the process according to the invention is suitable as the alcohol component for carrying out the process according to the invention. The term “alcohol component” as used here includes the alcohol in its protonated form, the alcohol in its deprotonated form, in particular salts of the alcohol, and also mixtures of the alcohol in its protonated form and its deprotonated form or mixtures of the alcohol in its protonated form, its deprotonated form and one or more salts of the alcohol.

Alcohols with a number of hydroxyl groups in a range of from 2 to 9, particularly preferably 3 to 8 and most preferably 3 to 6 are preferably employed as the alcohol component with one or more hydroxyl groups. The number of carbon atoms in the alcohol with one or more hydroxyl groups is preferably in a range of from 3 to 30, particularly preferably 3 to 18, furthermore preferably 3 to 10 and most preferably 3, 4, 5, 6, 7 or 8.

A technical grade alcohol can also be employed as the alcohol component. “Technical grade” in connection with a chemical substance or chemical composition means that the chemical substance or the chemical composition contains small amounts of impurities. In particular, the chemical substance or the chemical composition can contain impurities in a range of from 5 to 20 wt. %, preferably from 5 to 15 wt. %, more preferably from 5 to 10 wt. %, based on the total amount of the chemical substance or chemical composition. Particularly preferably, the chemical substance or the chemical composition contains from 1.5 to 5 wt. % of impurities. Impurities are understood as meaning all contents which differ from the chemical substance or the chemical composition. For example, technical grade ethanol can contain from 5 to 8 wt. % of impurities. This example cannot be generalized for all alcohols, rather the content of impurities with respect to the classification as “technical grade” is substance- or composition-related, or also depends on the preparation process. This classification according to the substance and the preparation process is familiar to the person skilled in the art.

It is likewise conceivable that it is not an individual alcohol or an individual technical grade alcohol which is employed as the alcohol component, but a mixture of several alcohols in the context of the abovementioned chemical composition. For example, several forms of the alcohol in accordance with that stated above can be employed as a mixture. Preferably, several alcohols characterized by at least one of the following features, such as different number of carbon atoms, different number of hydroxyl groups or different structure, or alcohols which differ simultaneously in two or more of the above-mentioned features, such as can be obtained, for example, as technical grade products from large-scale industrial processes, are employed.

According to the invention, the alcohol component comprises a polyol which is solid at 25° C. According to a preferred embodiment, difunctional, trifunctional, tetrafunctional or pentafunctional alcohols, or a mixture of two or more of these, are suitable as the alcohol component.

The following are suitable, for example, as the alcohol component based on difunctional alcohols: 2,3-butanediol, 1,6-hexanediol, 2,5-hexanediol, 3,4-hexanediol, 1,2-hexanediol, 1,2-cyclohexanediol, 1,3-cyclohexanediol, 1,2-heptanediol, 1,7-heptanediol, 2,6-heptanediol, 3,4-heptanediol, 1,2-cycloheptanediol, 1,3-cycloheptanediol, 1,4-cycloheptanediol, 1,2-octanediol, 1,8-octanediol, 2,7-octanediol, 4,5-octanediol, 1,2-cyclooctanediol, 1,3-cyclooctanediol, 1,4-cyclooctanediol, 1,5-cyclooctanediol, 1,2-nonanediol, 1,9-nonanediol, 2-methyl-1,9-octanediol, 2,2-dimethyl-1,9-octanediol, or two or more of these.

The following are suitable as the alcohol component based on trifunctional alcohols: erythrose, threose, trimethylolethane, trimethylolpropane, 2-hydroxymethyl-1,3-propanediol or two or more of these.

The following are suitable as the alcohol component based on tetrafunctional alcohols: erythritol, threitol, pentaerythritol, arabinose, ribose, xylose, ribulose, xylulose, lyxose, ascorbic acid, gluconic acid γ-lactone, or two or more of these.

The following are suitable as the alcohol component based on pentafunctional and more highly functional alcohols: arabitol, adonitol, xylitol and dipentaerythritol.

According to a further preferred embodiment, the alcohol component is chosen from pentaerythritol, pentaerythritol dimer, pentaerythritol trimer, trimethylolpropane, bistrimethylolpropane, pentaerythritol, pentaerythritol dimer, or two or more of these.

In this connection, reaction products of these alcohol components with ethylene oxide and/or propylene oxide which are solid at 25° C. are furthermore suitable.

According to a further preferred embodiment, the alcohol component contains less than 10 wt. %, preferably less than 5 wt. % of nitrogen-containing compounds, based on the total weight of the alcohol component, nitrogen-containing compounds being both nitrogen-containing alcohol components and other nitrogen-containing organic compounds. The alcohol component furthermore preferably does not contain nitrogen atoms (N).

According to a further preferred embodiment, the alcohol component contains less than 10 wt. %, preferably less than 5 wt. % of aromatic ring compounds, based on the total weight of the alcohol component, aromatic ring compounds being both alcohols containing aromatic rings and other aromatic ring compounds. The alcohol component furthermore preferably does not contain aromatic ring compounds.

According to a further preferred embodiment, the alcohol component contains as non-metal atoms only non-metal atoms chosen from the group consisting of carbon (C), oxygen (O), nitrogen (N) or hydrogen (H) or several of these, preferably consisting of carbon (C), oxygen (O) and hydrogen (H).

In principle all carboxylic acids known to the person skilled in the art can be employed as the carboxylic acid component for the preparation of the ester, the carboxylic acid component comprising a mono- or polyunsaturated, aliphatic carboxylic acid to the extent of at least 50 wt. %, the percentages by weight being based on the total amount of carboxylic acid components. The term “carboxylic acid component” as used herein includes the carboxylic acid in its protonated form, the carboxylic acid in its deprotonated form, and also salts of the carboxylic acid, and also mixtures of at least two of the above, or of the carboxylic acid in its protonated form, its deprotonated form and at least one or more salts of the carboxylic acid.

The term “carboxylic acid component” furthermore in principle includes all compounds which contain at least one carboxylic acid group. This term also includes compounds which, in addition to the at least one carboxylic acid group, contain other functional groups, such as ether groups.

A technical grade carboxylic acid can furthermore also be employed as the carboxylic acid component. It is likewise conceivable that it is not an individual carboxylic acid or an individual technical grade carboxylic acid which is employed as the carboxylic acid component, but a mixture of several carboxylic acids. For example, several forms of the carboxylic acid in accordance with that stated above can be employed as a mixture. Preferably, in the case of a mixture of several carboxylic acids the mixture is characterized by at least one, or also several, of the following features:
a varying number of carbon atoms,
a varying number of carboxyl groups, or
a varying structure.

Such mixtures which can be obtained as technical grade products from large-scale industrial processes often vary in several of the abovementioned features. Saturated carboxylic acids can also be present here as the carboxylic acid component as long as, as described according to the invention, at least 50 wt. % of unsaturated carboxylic acid, based on the total amount of all the carboxylic acid components, is present. The substance-related amount of impurities in the technical grade is familiar to the person skilled in the art.

The use of monocarboxylic acids is preferred according to the invention.

Possible carboxylic acid components are, in particular, unsaturated carboxylic acids or acid chlorides of unsaturated carboxylic acids and acid anhydrides of unsaturated carboxylic acids having a number of carbon atoms in a range of from 6 to 26, particularly preferably in a range of from 8 to 24, still more preferably in a range of from 10 to 22, moreover preferably in a range of from 12 to 20 and most preferably in a range of from 14 to 18, the entirety of the carboxylic acid components containing mono- or polyunsaturated, aliphatic carboxylic acid components to the extent of at least 50 wt. %. The carboxylic acid components furthermore preferably have 14, 16 or 18 C atoms.

Carboxylic acid components which are suitable in this connection are derived, for example, from the following unsaturated carboxylic acids, such as e.g. acrylic acid, methacrylic acid, 3-butenoic acid, 4-pentenoic acid, 5-hexenoic acid, 6-heptenoic acid, 7-octenoic acid, 8-nonenoic acid, 9-decenoic acid, undecylenic acid, palmitoleic acid, oleic acid, elaidic acid, vaccenic acid, icosenic acid, gadoleic acid, petroselic acid, ricinoleic acid, vernolic acid, cetoleic acid, erucic acid, and polyunsaturated carboxylic acids, for example linoleic acid, α-linolenic acid, arachidonic acid, timnodonic acid, punicic acid, α-elostearic acid, clupanodonic acid or cervonic acid.

According to a preferred embodiment, the carboxylic acid component is chosen from octanoic acid, i-octanoic acid, nonanoic acid, i-nonanoic acid, 9-decenoic acid, decanoic acid, i-decanoic acid, palmitic acid, stearic acid, oleic acid, pelargonic acid, behenic acid or erucic acid, or a mixture of at least two of these.

According to a further preferred embodiment, the carboxylic acid components in each case contain exactly one carboxyl group.

According to a further preferred embodiment, the carboxylic acid component contains less than 10 wt. %, preferably less than 5 wt. % of nitrogen-containing compounds, based on the total weight of the carboxylic acid component, nitrogen-containing compounds being both nitrogen-containing carboxylic acids and other nitrogen-containing organic compounds. The carboxylic acid component furthermore preferably does not contain nitrogen atoms (N).

According to a further preferred embodiment, the carboxylic acid component contains less than 10 wt. %, preferably less than 5 wt. % of aromatic ring compounds, based on the total weight of the carboxylic acid component, aromatic ring compounds being both carboxylic acids containing aromatic rings and other aromatic ring compounds. The carboxylic acid component furthermore preferably does not contain aromatic ring compounds.

According to a further preferred embodiment, the carboxylic acid component contains less than 10 wt. %, preferably less than 5 wt. % of compounds containing hydroxyl groups, based on the total weight of the carboxylic acid component, compounds containing hydroxyl groups being both hydroxycarboxylic acids, and other organic compounds containing hydroxyl groups. The carboxylic acid component furthermore preferably does not contain hydroxyl groups.

“Pure” and “technical grade oleic acid” can be employed as oleic acid. A pure oleic acid is understood as meaning a composition which contains more than 98 wt. % of oleic acid. A “technical grade oleic acid” is understood as meaning a composition which contains oleic acid to the extent of 98 wt. % or less. Such a technical grade oleic acid contains e.g. oleic acid in a range of from 60 to 75 wt. %, linoleic acid in a range of from 5 to 20 wt. % and stearic acid in a range of from 0 to 5 wt. %, based on the total weight of the technical grade oleic acid, the sum of the percentages by weight being 100. A suitable technical grade oleic acid is marketed e.g. by Cognis Oleochemicals GmbH, Germany, under the name “Edenor TiO5”. Such a technical grade oleic acid which can preferably be employed can be obtained from animal fats, for example beef tallow. A technical grade oleic acid with a higher content of oleic acid can likewise be employed, e.g. with 80 to 95 wt. %, preferably 85 to 95 wt. % and furthermore preferably 90 to 95 wt. %, in each case based on the total composition. A technical grade oleic acid with 96 to 98 wt. % of oleic acid, based on the total composition, is very particularly preferred. Another technical grade oleic acid with approx. 80 to 90 wt. % of oleic acid, 2 to 10 wt. % of linoleic acid, 2 to 6 wt. % of stearic acid and 2 to 6 wt. % of palmitic acid, based on the total weight of the other technical grade oleic acid, is furthermore preferred, the sum of the percentages by weight being 100. Such another technical grade oleic acid is marketed e.g. as “high oleic” sunflower oil or HO sunflower oil.

According to a further preferred embodiment, a partial ester is prepared as the ester, wherein
pentaerythritol, pentaerythritol dimer or a pentaerythritol oligomer, or two or more of these, is chosen as the alcohol component, and
a carboxylic acid mixture comprising at least 50 wt. % of oleic acid is chosen as the carboxylic acid component,
   a molar ratio of carboxylic acid groups of the carboxylic acid component to alcohol groups of the alcohol component of from 0.2 to 0.8 being established.

The term “partial ester” as used herein describes an ester of at least one carboxylic acid component and at least one alcohol component, where either
α.1) some of the hydroxyl groups of the alcohol component, for example in a range of from 20% to 80%, still more preferably from 25% to 75%, moreover preferably from 30% to 70%, still more preferably from 40% to 60%, and most preferably from 45% to 55% of the hydroxyl groups originally present in the alcohol component are still present as hydroxyl groups after the esterification reaction, or
α.2) some of the carboxyl groups of the carboxylic acid component, in particular if this comprises carboxylic acids with at least two carboxyl groups, for example in a range of from 20% to 80%, still more preferably from 25% to 75%, moreover preferably from 30% to 70%, still more preferably from 40% to 60%, and most preferably from 45% to 55% of the carboxyl groups originally present in the carboxylic acid component are still present as carboxyl groups after the esterification reaction and consequently are not esterified.

The term “full ester” accordingly describes an ester from the acids of the at least one carboxylic acid component and the at least one alcohol component, in which either
β.1) less than 20%, more preferably less than 10%, still more preferably less than 5%, moreover preferably less than 3%, still more preferably less than 2%, still more preferably less than 1% and most preferably less than 0.5% of the hydroxyl groups originally present in the alcohol component are still present as hydroxyl groups after the esterification reaction, or
β.2) less than 20%, more preferably less than 10%, still more preferably less than 5%, moreover preferably less than 3%, still more preferably less than 2%, still more preferably less than 1% and most preferably less than 0.5% of the carboxyl groups originally present in the carboxylic acid component are still present as carboxyl groups after the esterification reaction.

According to a further preferred embodiment, pentaerythritol dioleates can be prepared from oleic acid as the carboxylic acid component and pentaerythritol as the alcohol component. In addition to pentaerythritol and pure oleic acid, technical grades thereof can also be employed as reactants in this process. If technical grades are employed, a product which contains at least 40, preferably at least 50, particularly preferably at least 60, and moreover preferably at least 70 wt. %, in each case based on this product, of pentaerythritol oleate is usually obtained.

According to a further preferred embodiment, the carboxylic acid component comprises an amount of less than 25 wt. %, in particular less than 20 wt. %, or less than 13 wt. %, of saturated carboxylic acids.

According to a further preferred embodiment, a ratio of unsaturated C₁₆-carboxylic acids to unsaturated C₁₈-carboxylic acids of from 1:5.0 to 1:80, in particular from 1:7 to 1:20 is present in the carboxylic acid component.

According to a further preferred embodiment, the carboxylic acid component comprises oleic acid, preferably technical grade oleic acid. Oleic acid which can be obtained from beef tallow, sunflowers or sunflower crops rich in oleic acid is still more preferable.

The process according to the invention for the preparation of an ester from a carboxylic acid component and an alcohol component can be carried out in the presence of further additives, for example one or more catalysts, stabilizers, antioxidants, viscosity regulators and mixtures thereof.

The process according to the invention is preferably carried out in the presence of a catalyst. In principle, any compound which is known to the person skilled in the art and appears to be suitable for catalysis of the esterifications according to the invention is suitable here as the catalyst.

Preferably, the catalyst, or a catalyst mixture, is employed in a range of from 0.0001 to 5 wt. %, preferably from 0.0005 to 4 wt. %, further preferably from 0.001 to 3.5 wt. %, moreover preferably from 0.004 to 3.0 wt. %, in each case based on the total amount of process components a. and b. Particularly preferably, the amount of catalyst added is in a range of from 0.006 to 2.5 wt. %, from 0.008 to 2.2 wt. %, from 0.01 to 2.0 wt. %, from 0.03 to 1.8 wt. %, from 0.05 to 1.6 wt. % or from 0.08 to 1.3 wt. %, in each case based on the total amount of process components a. and b. A range of from 0.01 to 1.2 wt. %, from 0.02 to 1.1 wt. %, from 0.03 to 1.0 wt. % or from 0.04 to 0.9 wt. %, in each case based on the total amount of process components a. and b., is still more preferable.

If the catalyst is a solid at room temperature, the catalyst is preferably present in the form of particles, for example in the ground form. A particle size in a range of from 30 μm to 2 mm, in particular from 30 μm to 1 mm is preferred here. In accordance with that stated above, preferably at least 40 wt. %, in particular at least 45 wt. %, particularly preferably at least 50 wt. %, and most preferably a range of from at least 40 wt. % to 60 wt. % of the particles, in each case based on the total weight of the catalyst, have a particle size in the ranges described above.

At least one compound chosen from the group consisting of proton donor or electron donor, or both, can advantageously be employed as the catalyst.

Suitable catalysts from the group of proton donors are, for example, sulphuric acid or phosphoric acid, aliphatic or aromatic sulphonic acids, such as methanesulphonic acids or benzenesulphonic acids, such as o- or m-toluenesulphonic acid, particularly preferably p-toluenesulphonic acid or methanesulphonic acid. It is likewise conceivable to employ fluorinated aliphatic or aromatic sulphonic acids, particularly preferably trifluoromethanesulphonic acid.

Suitable catalysts from the group of electron donors are, preferably, metals, metal compounds or reducing acids. Suitable metals are, in particular, tin, titanium, zirconium, which are preferably employed as finely divided metal powders. Suitable metal compounds are the salts, oxides or soluble organic compounds of the metals described above, or a mixture of at least two of these. In contrast to the proton donors, the metal compounds are high temperature catalysts which as a rule achieve their full activity only at temperatures above 180° C. They are preferred according to the invention because a smaller amount of by-products, such as, for example, olefins, are formed compared with catalysis with proton donors. Catalysts which are particularly preferred according to the invention are a) one or more divalent tin compounds, or b) one or more tin compounds and elemental tin, which can react with the reactants to give divalent tin compounds. For example, tin, tin(II) chloride, tin(II) sulphate, tin(II) alcoholates, or tin(II) salts of organic acids, in particular of mono- and dicarboxylic acids, e.g. dibutyltin dilaurate, dibutyltin diacetate, or a mixture of at least two of these, can be employed as the catalyst. Particularly preferred tin catalysts are tin(II) oxalate and tin(II) octoate.

In principle all reducing acids which are known to the person skilled in the art and appear to be suitable are suitable as catalysts of the group of reducing acids. Hypophosphorous acid, sulphurous acid, selenious acid, oxalic acid, ascorbic acid, or two or more of these are particularly preferred.

According to a further preferred embodiment, a mixture which comprises at least two, in particular at least three catalysts from one or more of the above-mentioned groups is employed as the catalyst. Particularly preferably, two or more catalysts are chosen, each catalyst being chosen from in each case different abovementioned groups.

According to a further preferred embodiment, a catalyst mixture comprising at least two different catalysts is employed, the first catalyst being chosen from the group of proton donors and the at least one further catalyst being chosen from the group of electron donors, or a mixture of two or more of these. Such a catalyst mixture can have a high catalytic activity at temperatures which are lower compared with the high temperature catalysts, e.g. between 140 and 180° C., or between 120 and 185° C. At the same time, because of the lower process temperature, a smaller amount of by-products which appear coloured, in particular a smaller amount of substances which cause a yellowish or brownish colouring, is formed. A mixture comprising p-toluenesulphonic acid and a tin compound is particularly preferred as the catalyst mixture. According to a further preferred embodiment, the catalyst or the catalyst mixture does not contain tin oxide.

According to a further preferred embodiment, the catalyst comprises tin oxalate, in particular in a range of from 0.01 to 5.0 wt. %, still more preferably from 0.01 to 0.08 wt. %, in each case based on the total weight of the sum of alcohol components and carboxylic acid components.

According to a further preferred embodiment, a mixture comprising 0.001 to 1 wt. % of an electron donor of the group of metal or metal compound, 0.001 to 1 wt. % of a proton donor and 0.001 to 1 wt. % of a second electron donor from the group of reducing acid, in each case based on the total amount of process components a. and b., can be employed as the catalyst. Particularly preferably, tin oxalate is chosen as the metal compound, p-toluenesulphonic acid is chosen as the proton donor and hypophosphorous acid is chosen as the reducing acid.

According to a further preferred embodiment, the pressure described above is applied at a point in time after the start of the reaction up to the end of the reaction. This pressure is particularly preferably applied after ⅓, ½, or ¾ of the duration of the reaction. The duration of the reaction is understood as meaning the period of time during which process step ii. is carried out.

According to a preferred embodiment, a pressure in a range of from 2 to 600 mbar, furthermore preferably 2 to 200 mbar, or 2 to 100 mbar, particularly preferably 2 to 50 mbar and most preferably 2 to 20 mbar is applied to the reactor during the entire duration of the reaction.

It is furthermore preferable to apply this pressure with a profile over time starting with 500 to 600 mbar and ending with 0.5 to 2 mbar, the pressure being kept at 0.5 to 2 mbar during the last 10%, the last 20%, the last third, or the second half of the time of the duration of the reaction.

In the context of carrying out the process according to the invention, process components a., b., d. and optionally c. are first employed in process step i. The sequence and the nature and manner of the addition of the individual components a., b., d. and optionally c. into the reactor in principle is not critical. Preferably, all the process components required for a reaction which are to be attributed to one of the groups chosen from alcohol component, carboxylic acid component and catalyst are in each case introduced into the reactor as process components within the particular group at least partly at the same time. In this context, the carboxylic acid components and alcohol components envisaged for the preparation of the ester according to the invention can be initially introduced and can then be reacted in the presence of a suitable catalyst or a suitable catalyst mixture. Furthermore, in a preferred embodiment the catalyst components are initially introduced together with one of the process components chosen from one of the groups of alcohol components or carboxylic acid components and the other components are then added. If the catalyst components are introduced into the reactor together with a process component, this can be effected by simultaneous introduction, and by introduction as a mixture, solution, suspension or dispersion.

Process components a., b., d. and the additives c. are provided in the reactor in liquid or in solid form. It may be preferable in the case of process components to be provided which are solid at the ambient temperature to be liquefied by heating. It is conceivable both that the liquefying is carried out in the course of providing the components, e.g. by means of a preheating stage, and that these process components are stored in liquid form at elevated temperature and are led from the holding place under thermostatic control and in an insulated line through a metering device. The addition of the process components in liquid form makes simple metering possible and promotes swift mixing of the process components introduced into the reactor.

Suitable metering devices are in principle all the devices which are known to the person skilled in the art and appear to be suitable. Electrically controllable shut-off valves or delivery pumps are particularly suitable.

The addition of the additives c. is in general carried out in a separate step to the components a., b. and d. already initially introduced. If these are solids, these are preferably introduced through a sluice at the upper side of the reactor, the contents of the reactor being stirred vigorously. A cellular wheel sluice can particularly preferably be employed as the sluice. It is often advantageous to mix the components, while stirring, in the context of providing them.

If at least one catalyst or a catalyst mixture is employed as an additive, a mixture of solids, a suspension or a liquid mixture is preferably employed. Preferably, the catalyst or the catalyst mixture is added only at the start of the reaction.

The reaction of the process components in process step ii. of the process according to the invention can be carried out by all processes which are known to the person skilled in the art and appear to be suitable. In this context, it may be advantageous to remove the water formed in the reaction from the reaction mixture, this removal of the water preferably being carried out by distillation during the reaction.

When the reaction has ended, in particular after the unreacted alcohol has been separated off, the catalyst present in the reaction mixture can furthermore be separated off by washing with water, a filtration or by centrifugation, optionally after treatment with a base.

It is furthermore preferable to carry out the reaction at a temperature in a range of from 50 to 300° C., particularly preferably in a range of from 100 to 250° C. and very particularly preferably in a range of from 100 to 280° C., most preferably in a range of from 150 to 250° C. and furthermore preferably in a range of from 200 to 250° C. The preferred temperatures depend on the alcohol component chosen, the progress of the reaction, the catalyst type and the catalyst concentration. These can be easily determined by experiments for each individual case. Higher temperatures increase the rates of reaction and promote side reactions, for example splitting off of water from alcohols or the formation of coloured by-products or both.

It is also preferable to carry out the reaction of the process components at a temperature in a range of from 50 to 160° C., particularly preferably in a range of from 80 to 150° C. and very particularly preferably in a range of from 100 to 140° C., most preferably in a range of from 120 to 140° C. Preferably, proton acids are then employed as the catalyst or catalyst mixture. Particularly preferably, no further catalysts are then added.

It is furthermore preferable to keep the process components uniformly mixed by stirring during the reaction.

According to a further preferred embodiment, a part of the process components is removed continuously from the reactor during the reaction, fed via a delivery line to an external flow-through heat exchanger and then fed back into the reactor. The external flow-through heat exchangers can be configured in any manner which is known to the person skilled in the art and appears to be suitable. Preferably, a plate heat exchanger, a tube bundle heat exchanger or a falling film evaporator or a combination of at least two of these, particularly preferably at least one falling film evaporator, can be employed as the flow-through heat exchanger. Furthermore, the outflow of the flow-through heat exchanger is connected to the reactor preferably via a return line of not more than 300 cm to 1 cm length, particularly preferably less than 200 cm to 10 cm length, most preferably less than 100 cm to 40 cm length. Particularly preferably, the outflow from the flow-through heat exchanger is connected directly, preferably via a flange, to the upper side of the reactor.

It is furthermore preferable in connection with the process according to the invention for the ester A obtained in the reaction ii. to be after-treated.

“After-treatment” is understood as meaning all conceivable steps and processes which are familiar to the person skilled in the art and which can be undertaken in order to purify the ester A obtained in process step ii. from by-products, impurities, catalysts and other additives or those processes with which the ester A is further processed to an end product.

These are understood as meaning, in particular, distillation, sorption, filtration, bleaching, centrifugation, washing, crystallization or drying processes, and continuing reactions, or a combination of at least two or more of these. Pressure filtration, bleaching and spray drying processes are preferred.

In the process according to the invention, the ester A is after-treated in a working up container with the following steps:
aa. provision of the ester A,
bb. addition of water in an amount of from 1 to 10 wt. %, based on the weight of the ester A,
cc. mixing to give an aqueous phase,
dd. separating off of the aqueous phase to give an ester B,
ee. optionally drying of the ester B,
ff. optionally treatment with a sorbent,
   the after-treatment being carried out at a temperature of 70-100° C.

For the after-treatment, the ester A is transferred according to the invention into a working up container. The ester A can be provided by any measure which is known to the person skilled in the art and appears to be suitable. Preferably, it is provided directly by a fluid-carrying connection from the reactor, or via an intermediate stage, which is preferably configured as a heat exchange zone. Such a heat exchange zone can be provided between the reactor and the working up container if the after-treatment is to be carried out at a temperature which differs from the temperature of the reaction. The temperature in the after-treatment is preferably in a range of from 10 to 200° C., particularly preferably from 50 to 100° C., further preferably from 60 to 90° C. and most preferably from 70 to 80° C. below the temperature of the reaction. The provision of such a heat exchange zone can considerably shorten the occupation time in the reactor.

According to step bb., water is added in an amount in a range of from 1 to 10 wt. %, based on the weight of the ester A. The ester A and the water are then mixed in step cc. The mixing can be carried out by any device which is known to the person skilled in the art and appears to be suitable in the present case and/or any process which is known and appears to be suitable. Reference is made here to the devices and processes which are disclosed at a later point in the description and are thus also suitable for mixing the ester A with water during the after-treatment.

On mixing of the ester A with water, an aqueous phase is formed, which is separated off in step dd. to give the ester B. The aqueous phase can be separated off in principle by any process which is known to the person skilled in the art and appears to be suitable. Those processes which are not associated with the introduction of additional heat into the ester A to be purified are particularly preferred. Processes such as e.g. decanting processes, phase separation are therefore particularly advantageous. Distillation processes are consequently less preferable in comparison.

According to a further preferred embodiment, steps aa. to dd. are repeated between twice and ten times, during the second and each further time in each case the ester B from step dd. of the preceding time being provided as ester A in step aa. Particularly preferably, the number of repetitions is 3 to 8, still more preferably 4 to 7, and most preferably 5 or 6.

It is furthermore preferable for the water to be added in step bb. to the ester A from step aa. via a distributing device. Any distributing device which is known to the person skilled in the art and appears to be suitable is suitable as the distributing device. One or more nozzles such as can be arranged, for example, on a spray ring are particularly suitable as the distributing device. The water can also be introduced and mixed via a deflecting surface, an atomizer, or by bringing together the substance flows of ester A and water with a flow divider and/or fluidizing unit arranged downstream, or several of these, e.g. in a system of tubes. Steps bb. and cc. can therefore also coincide, at least partly.

According to a further preferred embodiment, the ester B is dried in step ee. at a temperature of from 90 to 150° C., preferably under a pressure in a range of from 2 to 600 mbar. Particularly preferably, the ester B is dried in step ee. at a temperature of from 90 to 130° C., or 90 to 120° C., or 90 to 110° C. Likewise preferably, a pressure of from 2 to 600 mbar, or 2 to 400 mbar, or most preferably from 2 to 200 mbar is applied here.

According to a further preferred embodiment, the ester B is treated further with a sorbent at a temperature in a range of from 70 to 100° C.

According to a further preferred embodiment, the ester B is combined in step ff. with a sorbent to give a mixture, before this mixture is divided into a solid phase and a liquid phase, the ester being obtained as the liquid phase.

In principle, any sorbent which is known to the person skilled in the art and appears to be suitable for the after-treatment can be employed as the sorbent. A mixture of two or more sorbents can likewise also be chosen. Sorbents in the context of the invention are understood as meaning in particular substances which can make a contribution towards improving the physical properties or the purity of the ester B prepared according to the invention or to both, without changing the identity of the ester B according to the invention by a chemical reaction. Preferably, the sorbent is introduced into the ester B as a particulate solid.

According to a preferred embodiment, an amount of the sorbent in a range of from 0.01 to 20 parts to 100 parts of process components is introduced into the ester B. Furthermore preferably, an amount of the sorbent in a range of from 0.05 to 10 parts, or from 0.1 to 5 parts, in particular from 0.1 to 3 parts, from 0.1 to 2 parts, or from 0.1 to 1 part, in each case to 100 parts of process components, is chosen. Still more preferably, an amount of the sorbent in a range of from 0.25 to 0.8 part, very particularly preferably in a range of from 0.3 to 0.7 part, most preferably from 0.4 to 0.6 part, that is to say, for example, 0.5 part, in each case to 100 parts of process components, is chosen. An amount of the sorbent in a range of from 0.3 to 1.0 part, in particular 0.4 to 0.8 part, in each case to 100 parts of process components, can occasionally also preferably be chosen.

In principle all particle sizes which are known to the person skilled in the art and appear to be suitable for the purpose of the present invention are possible as particle sizes of the sorbent introduced as a particulate solid. The solid is described as particulate in particular if at least some of its particles have a particle size of from 8 μm to 5 mm.

In accordance with that stated above, preferably at least 40 wt. %, in particular at least 45 wt. %, particularly preferably at least 50 wt. %, and most preferably a range of from at least 40 wt. % to 60 wt. % of the particles, in each case based on the total weight of the sorbent, have a particle size in a range of from 8 μm to 0.1 mm. The percentage by weight data described in the above sentence likewise in each case apply to the following particle size ranges: from 20 μm to 300 μm, or preferably from 16 to 72 μm, or from 20 μm to 50 μm, in each case based on the total weight of the sorbent.

The sorbent present as a particulate solid can have particles of a single particle size, or particles of several particle sizes which form a particle size distribution. If a particle size distribution is present, a distribution which resembles a bell-shaped curve or corresponds to this is preferred. The particle size distribution, which is also called grain size distribution, is particularly preferred, the maximum of the grain size distribution which is preferred according to the invention being in a range of from 15 to 75 μm, preferably 20 to 60 μm, or 25 to 50 μm.

It is furthermore preferable for the particles to have pores, preferably those with a preferred pore size in a range of from 5 to 10 μm. Pore size is understood as meaning the arithmetical average diameter of a pore, in a range of from 50% to 99.9%, preferably from 70% to 99.9% and most preferably from 85% to 99.9% of the pores having this diameter.

It is furthermore also possible for agglomerates of particles to occur if two or more particles stick to one another. Such agglomerates are also included in the particle term according to the invention, regardless of the composition and formation thereof.

Furthermore, in one embodiment of the present invention the sorbent is chosen such that its fine dust content is as low as possible. The fine dust content is understood as meaning that weight content of particles which has a particle size of less than 8 μm. Preferably, the fine dust content is less than 30 wt. %, less than 20 wt. %, less than 10 wt. %, preferably less than 5 wt. %, or less than 0.5 wt. %, in each case based on the total weight of the sorbent. The fine dust content is often in a range of from 1 to 10 wt. %, based on the total weight of the sorbent.

According to a further preferred embodiment of the present invention, the sorbent has a BET surface area according to DIN 66131 in a range of from 0.5 to 1,500 m²/g.

Substances chosen from the group of inorganic silicon-oxygen compounds, active charcoal, kieselguhr, ion exchangers, or two or more of these are suitable, for example, as the sorbent. Kieselguhr is preferably chosen.

In the context of the present invention, the term “active charcoal” also includes active charcoal-carbon black, active charcoal-coke and graphite. Occasionally, however, non-active charcoal molecular sieves are employed as “active charcoal”. According to the invention, an active charcoal which is preferably chosen is one which consists to the extent of more than 80 wt. %, or more than 90 wt. %, or more than 95 to 99 wt. % of carbon, particularly preferably of elemental carbon. Such an active charcoal is particularly advantageous if it has a BET surface area in a range of from 800 to 1,100 m²/g, furthermore preferably from 850 to 1,050 m²/g, or from 900 to 1,050 m²/g, or from 900 to 1,000 m²/g.

In addition to a direct use as a sorbent, active charcoal can also be employed in combination with a further sorbent, or on a carrier material, or both. If the active charcoal is employed in combination with at least one further sorbent, the content of active charcoal can vary within a wide range, e.g. between 0.1 and 90 wt. %, based on the total weight of the sorbents. Preferably, the content of active charcoal is between 0.5 and 70 wt. %, in particular between 5 and 40 wt. %, based on the total weight of the sorbents. A uniform homogeneous distribution of the active charcoal is particularly advantageous.

Furthermore, sorbents coated with active charcoal can also be employed as the active component. In this case the active charcoal is at least partly combined with a carrier material. The carrier material can be either amorphous or crystalline or present in a mixed form of the two. Oxidic material can furthermore preferably be employed as the carrier material. Amorphous, preferably amorphous oxidic carrier materials, which can contain up to 50 wt. %, preferably 2 to 10 wt. %, based on the carrier material, of crystalline material, are particularly preferred. If crystalline contents, e.g. zeolite or aluminium phosphate, are present, the content thereof is advantageously in the range of from 0.5 to 50 wt. %, based on the total weight of the sorbents.

Inorganic silicon-oxygen compounds, preferably a silicate, or two or more of these are suitable as the sorbent. The inorganic silicon-oxygen compound advantageously has a BET surface area of from 150 to 240 m²/g, particularly preferably from 180 to 220 m²/g, for example 195m²/g.

One or more compounds chosen from the group consisting of: silica, in particular in disperse or highly disperse form, kieselguhr, clay mineral, in particular montmorillonite or bentonite, or zeolites, or two or more of these, are preferably employed as the silicate.

Kieselguhr or bentonite, or both, are particularly preferably employed as the inorganic silicon-oxygen compound. Calcium bentonite is particularly preferably suitable as the bentonite, very particularly preferably acid-activated calcium bentonite.

A combination of two or more sorbents, in particular a combination of at least one inorganic silicon-oxygen compound and at least one active charcoal, or two or more of these, can furthermore be employed. If such a combination of at least one inorganic silicon-oxygen compound and at least one active charcoal is employed, a ratio of inorganic silicon-oxygen compound to active charcoal in a range of from 10:1 to 1:10, or from 5:1 to 1:5, or from 5:1 to 1:1, particularly preferably from 4:1 to 1.5:1 is advantageously employed. A combination of inorganic silicon-oxygen compound to charcoal compound in a range of 3:1 to 2:1 is very particularly preferred.

If kieselguhr is chosen as the sorbent, kieselguhr with a BET surface area of from 0.5 to 7 m²/g is preferred. Kieselguhr with a weight-average particle size of from 10 to 50 μm, or from 20 to 40 μm is further preferred. The particle size can be determined with a Leeds & Northrup “X100 Microtrac particle size analyzer”.

According to a preferred embodiment, further auxiliary substances can be introduced into the working up container for after-treatment of the ester A. All substances which are known to the person skilled in the art and appear to be suitable can be chosen as auxiliary substances for this.

Suitable auxiliary substances are, for example, antistatics, antioxidants, anticaking agents, trickle agents, inhibitors, desiccants, rheology modifiers, or two or more of these.

A mixture of two or more of the abovementioned auxiliary substances which are assigned to the same or different abovementioned groups of auxiliary substances can furthermore be employed.

It is furthermore conceivable for the ester A, the ester B, the sorbent or the auxiliary substance, or several of these, to have a content of a liquid. With respect to the liquid-containing substance, this liquid can have been introduced due to the preparation, from the environment or intentionally incorporated into the substance, e.g. in order to avoid release of dust during handling of a particulate solid. This is carried out, for example, if the sorbent is employed as a suspension.

According to a further preferred embodiment, the sorbent is introduced as a particulate solid with less than 5 wt. % of a liquid, based on the sorbent, into the ester B.

The substances transferred and introduced into the working up container are combined to form a mixture. This can be carried out in principle in any manner which is known to the person skilled in the art and appears to be suitable. For example, the substances can be mixed with a stirrer, by pumping the mixture in circulation or by introducing a gas into the lower region of the working up container. The mixture obtained in this way can be homogenized by further mixing, for example stirring.

Thereafter, the mixture is divided into a solid and a liquid phase, the ester being obtained as the liquid phase. Any method which appears to be suitable to the person skilled in the art can be employed for dividing the mixture. Filtration, pressure filtration, settling or centrifugation processes are preferred. Pressure filtration processes are particularly preferred. A pressure filtration process is understood as meaning such a process in which a mixture to be filtered is charged with pressure and is led into a filter device and divided on a filter surface, for example a narrow-mesh net, a filter paper, a woven fabric or a laid fabric. A filter press is preferred as the filter device. A pressure filtration process which is preferred according to the invention can be carried out under a pressure in a range of from 0.5 to 20 bar, preferably from 1 to 10 bar, furthermore preferably from 1.5 to 8 bar, likewise preferably from 2 to 7 bar, from 2.5 to 5 bar and most preferably in a range of from 3 to 4 bar. The pressure filtration process is often also carried out under a pressure in a range of from 1 to 3 bar.

It is furthermore preferable according to the invention to carry out the division in the separating device at a temperature of from 60 to 100° C., preferably from 70 to 90° C. and most preferably from 85 to 90° C. The liquid phase thereby formed can be fed directly to a further process step, or at least partly fed back into the working up container in a circulation. Preferably, the after-treatment is carried out in a circulation for a certain duration. For example, the after-treatment can be carried out with a dwell time in a range of from 15 to 240 minutes, preferably from 30 to 120 minutes, further preferably from 40 to 90 minutes and particularly preferably from 50 to 60 minutes. A dwell time of from 40 to 90 minutes is preferred.

It may moreover be advantageous to carry out the after-treatment under increased pressure, at elevated temperature and over a certain dwell time, or under a combination of two or more of the abovementioned conditions.

In the context of the pressure filtration process described here, for the division the mixture can be led through a separating device with one or more filter chambers, the mixture being divided into a solid and a liquid phase in the one or the several filter chambers on one or more filter surfaces. Preferably, the separating device has at least two filter chambers. Particularly preferably, the separating device has in a range of from two to 50, further preferably in a range of from 5 to 30, or from 10 to 25, or from 15 to 20 filter chambers.

During the division of the mixtures, in the separating device a solid phase with a thickness in a range of from 1 to 20 mm, preferably from 2 to 18 mm, or from 4 to 15 mm, or from 5 to 12 mm, or from 6 to 10 mm or from 7 to 8 mm often forms in at least one of the filter chambers on at least one of the filter surfaces. Such solid phases can also be formed in several filter chambers or on several filter surfaces, or both. Preferably, solid phases are formed on all the filter surfaces in all the filter chambers of the separating device. It is furthermore conceivable for the mixture itself to be divided into at least two streams before the division into a solid and a liquid phase, for each stream to be divided into a liquid and a solid phase in its own separating device, and for the liquid phases obtained in this way then to be brought together again.

The solid phase preferably comprises the sorbent, preferably in an amount in a range of from 30 to 90 wt. %, further preferably from 50 to 80 wt. %, based on the total amount of solid phase.

According to a further preferred embodiment, the ester B as the ester has at least one, preferably several, or all of the following features:
a Gardner colour number of 7, or less,
a water content of less than 0.1 wt. %, in particular of from 0.01 to 0.05 wt. %, in each case based on the total weight of the ester,
an acid number of from less than 2 to more than 0.1, in particular from less than 1.5 to 1.0,
a hydroxyl group number (also: OH number, OHN) in a range between 90 and 200, in particular from 100 to 150,
a melting point in a range of from −50 to −10° C., in particular −35 to −15° C.,
a cloud point in a range of from −20 to −5° C., in particular −10 to −0° C.

When the after-treatment has ended, the ester B can be collected and made available in a holding unit.

In connection with the esters which can be prepared from a carboxylic acid component and an alcohol component with several hydroxyl groups in the process according to the invention, it is furthermore preferable for not all the hydroxyl groups of the alcohol component to be esterified, so that some of the hydroxyl groups remain non-esterified. In this connection, it is particularly preferable for from 5 to 80 mol %, particularly preferably from 10 to 70 mol %, still more preferably from at least 20 to 50 mol %, moreover preferably from 30 to 40 mol % and most preferably from 45 to 55 mol % of the hydroxyl groups of the alcohol component not to be esterified. This means that in the ester obtainable by reaction of the composition according to the invention, the content, described in mol %, of all the hydroxyl groups originally present in the alcohol component containing several hydroxyl groups for the preparation of the ester from a carboxylic acid component and an alcohol component is not esterified, and thus is also present as hydroxyl groups in ester A, and optionally also in ester B.

The present invention also provides a device comprising as device units connected by fluid-conducting means
α) at least one reactant reservoir,
β) a reactor with a mixing device,
γ) a working up unit,
   wherein the working up unit comprises, connected by fluid-conducting means:
αα) a working up container,
ββ) a delivery pump and
γγ) a separating device,
   wherein a filter press which has 2 or more filter chambers is employed as the separating device, at least two of these filter chambers being provided with a filter frame, and each filter frame being provided with a filter material, the filter material having a permeability to air of from 5 to 20 l·m⁻²·s⁻¹ and a weight per unit area of from 500 to 700 g/m².

In principle, all reactor types known to the person skilled in the art which this person considers suitable for carrying out the process according to the invention can be employed. Preferably, a stirred tank on the side wall of which is arranged a heating jacket on the outside or inside is employed as the reactor. The heating jacket can be arranged on a part of the side wall or on the entire side wall. Preferably, the heating jacket is arranged on the entire side wall. Furthermore, the heating jacket particularly preferably can be controlled in sections. For example, the heating jacket is in 3, 4, 5 or more sections, each of which can be heated independently of each other. For transportation of heat, a heat transfer medium is led to the heating jacket through heating lines. All the usual heat transfer media known to the person skilled in the art are suitable as the heat transfer medium. The heat transfer medium can be either a heating means or a coolant. The heat transfer medium can also be under pressure. Preferably, heating steam, thermal oil or water, particularly preferably heating steam, is chosen as the heat transfer medium.

Furthermore, the reactor advantageously has a stirrer with a stirrer motor, transmission and stirrer shaft with stirrer blades, which is arranged on the upper side of the stirred tank, preferably centrally. The length of the stirrer shaft, the number of stirrer tiers arranged on the stirrer shaft, the diameter of these stirrer tiers and the geometry of the stirrer blades arranged in each stirrer tier are advantageously chosen such that during operation a uniform mixing of the process components, and where appropriate of the reaction products, is ensured, especially in the regions close to the base. The length of the stirrer shaft is preferably chosen such that the stirrer shaft extends from a motor lying outside the reactor, or from a transmission driven by a motor, almost to the base of the reactor. Preferably, the length of the stirrer shaft is chosen such that a distance of between about 5 to about 10%, with respect to the height of the reactor tank, remains between the end of the shaft and the reactor base. The stirrer shaft can be mounted on one side or, if the stirrer shaft is constructed to the reactor base, mounted at two points.

All stirrer types known to the person skilled in the art which this person considers suitable for carrying out the process according to the invention can be employed as stirrers. Preferably, stirrer types which have the effect at least in part of axial mixing during operation can be employed in particular. The stirrers can have one or more stirrer tiers, preferably one, 2, 3, 4, 5, 6 or 7 tiers. With respect to the geometry, cross, angled blade or disc stirrers with suitable stirrer blades are particularly preferred, and MIG or INTERMIG stirrers are most preferred. In the case of angled blade, disc and MIG stirrers, the stirrer blades in adjacent tiers can be displaced by 90° in the horizontal plane. The stirrers particularly preferably have an even number of tiers.

The stirrers are preferably produced from steel, preferably from V2A or V4A steel, in particular from the following materials, the material numbers being found in EN10088: 1.4307, 1.4306, 1.4311, 1.4301, 1.4948, 1.4404, 1.4401, 1.4406, 1.4432, 1.4435, 1.4436, 1.4571, or 1.4429, particularly preferably 1.4301 or 1.4571.

The stirrer can moreover be at least partly coated with a surface coating composition. Preferably, the stirrer is equipped with a polymer coating. For example, a fluoropolymer coating which protects the material from which the stirrer is made from the fluid or mixture to be stirred is suitable as a polymer coating.

Preferably, a ratio of the diameter of the stirrer tier(s) to the diameter of the reactor of from 0.55 to 0.75, particularly preferably 0.60 to 0.70 or 0.62 to 0.68, very particularly preferably 0.64 to 0.66, e.g. 0.65, is chosen. By suitable choice of the parameters, the person skilled in the art ensures complete mixing and mingling in the reactor and avoids a deposit of solid constituents.

The stirrer blades can have the most diverse geometries, the geometry influencing the nature of the mixing. The “nature of the mixing” is understood as meaning the polar vector acting on the stirred mixture due to the movement of the stirrer. The polar vector has vertical and horizontal contents. Usually, both contents are not equal to zero. For example, a cross stirrer with stirrer blades arranged axially to the stirrer shaft and aligned vertical to the stirrer plane has the effect of rather horizontal mixing, whereas a cross stirrer with stirrer blades arranged at an angle, e.g. axially, to the stirrer shaft and at an angle of 30°, 45° or 60° with respect to the stirrer plane has the effect of a more vertical mixing. It is furthermore conceivable to provide a spiral stirrer.

Stirrers of which the stirrer blades have, with respect to the stirrer plane, a positive incline in the region close to the stirrer shafts, preferably the inner two thirds of the stirrer blade, and a negative incline in the region away from the stirrer shafts, preferably the outer third of the stirrer blade, are particularly preferred.

The incline of a stirrer blade is understood as meaning its alignment with respect to the stirrer plane, a positive incline meaning that the stirrer blade rises in the direction of rotation from its front edge from the bottom upwards to its rear edge, and has the effect of an ascending flow of material. A negative incline means that the stirrer blade drops in the direction of rotation from its front edge from the top downwards to its rear edge, i.e. has the effect of a descending flow of material. Such a stirrer has the effect of vertical mixing from the bottom upwards in the region of the middle of the reactor and a vertical mixture from the top downwards at the reactor wall.

The type of mixing described above can be assisted and adapted with further auxiliary devices. For example, baffles can be provided on the inside wall of the reactor. These are preferably attached to the inside wall of the reactor in the vertical direction, the plane in which the baffle lies being aligned through or at least in the direction of the vertical axis of the reactor.

According to another example, end stirrer organs which are moved a short distance above the reactor base can be attached on the lower stirrer tier. A short distance is to be understood as meaning so small that deposits of solid on the bottom can be carried along and/or swirled up by the stirrer. In this context, the end stirrer organs have the effect of a horizontal mixing to the extent of at least 50%, preferably 70%, based on the layer mixed by the end stirrer organs. The end stirrer organs preferably have a flat shape, the sides of the flat shape which are adjacent to the reactor base and the reactor wall being designed such that an essentially constant gap is provided between the reactor base and the reactor wall. If the reactor base is curved, for example, the end stirrer organs have a surface which is at least rounded at the side, and optionally an angled position of the end stirrer organs. Preferably, the end stirrer organs sweep over the reactor base at a distance of from 10 to 30 cm, preferably 15 to 25 cm or 30 cm.

In principle all materials which are known to the person skilled in the art and which this person considers suitable with respect to carrying out the process according to the invention, in particular with respect to strength, elasticity and corrosion resistance, can be employed as the material for production of the devices described above. In particular, the materials which are preferred in the choice of material for the stirrer are also preferred. Preferably rustproof steel, in particular V2A or V4A steel, preferably from V2A or V4A steel, in particular from the following materials, is employed for production of the reactor, the material numbers being found in EN10088: 1.4307, 1.4306, 1.4311, 1.4301, 1.4948, 1.4404, 1.4401, 1.4406, 1.4432, 1.4435, 1.4436, 1.4571, or 1.4429, particularly preferably 1.4301 or 1.4571.

The device furthermore has a working up unit. Any device which is known to the person skilled in the art and appears to be suitable for improving a certain parameter of the crude product obtained in the reaction can be conceived as the working up unit. For example, a purification or separating device can be provided as the working up unit. Devices which have both a purifying and a separating action are usual in particular. Distillation units, filters, filter presses, sieves, separators, clarification devices or centrifuges, or a combination of two or more of these, are preferably suitable as working up units.

A line for removing a gaseous fluid stream which, for example, can remove by-products with a molecular weight of less than 100 g/mol is furthermore preferably provided on the reactor, this line being connected, if desired, to a pressure reducing unit for applying a reduced pressure. The fluid stream can furthermore be further treated, and for this led over at least one heat exchanger in order to cool the fluid stream. In this context, at least a part of the fluid stream can pass into a liquid phase, which is often collected and led back into the reactor or removed. In this context, at least a part of the fluid stream can pass into a liquid phase, which is often collected and led back into the reactor or removed. This treatment of the fluid stream can be repeated twice or more often. If the fluid stream is led over at least two heat exchangers arranged in series, in the first heat exchanger a part of the fluid stream which differs from that in the at least second heat exchanger can pass into a liquid phase. It is thus possible, if desired, to lead a part of the fluid stream back into the reactor as a liquid phase and to discard another part of the fluid stream. Furthermore, the part of the fluid stream which is to be led back into the reactor as a liquid phase can optionally be divided into two immiscible phases in a separator with the aid of an adjustable removal device. Such a removal device is configured, for example, as an interfacial layer regulator. The first phase can then be passed back into the reactor via a return line. Alternatively, the entire fluid stream can be drained off and e.g. fed to another use, or discarded. The division of the fluid in the separator into two immiscible phases is carried out by appropriate alignment of the interfacial layer regulator. In principle any known embodiment which appears to be suitable to the person skilled in the art is suitable as the interfacial layer regulator.

It is furthermore conceivable to collect the fluid stream in a receiver before introduction into the separator, to lead the fluid stream over an additional heat exchanger and to further cool it in this way. At a lower temperature of the fluid stream, a better and faster demixing of at least two immiscible, liquid phases can be observed.

A heat transfer medium is led through each of the heat exchangers already mentioned. In order to effect cooling of the fluid stream, cooling fluids are preferably employed as heat transfer media. Preferably, the highest possible temperature difference is chosen between the fluid stream to be liquefied and the cooling fluid, in order to achieve marked cooling of the fluid stream. Furthermore, it may be entirely desirable to cool the fluid stream in a first step merely to a first temperature at which a part of the fluid stream is liquefied, before a further part of the fluid stream is liquefied in a downstream heat exchanger. It is conceivable that a first heat exchanger is operated with a cooling fluid which, for example, is at 20 or 25° C., or has a higher temperature, in order to separate off from the fluid stream at least a high-boiling content of the fluid stream which, e.g. has a boiling point in a range of from 80 to 120° C.

In this connection, a high-boiling content is understood as meaning one or more components of the fluid stream which have a boiling point in a range of from 50 to 150° C., preferably from 60 to 140° C., very particularly preferably from 70 to 130° C. In particular, a high-boiling content is understood as meaning those components which have a boiling point of from 80 to 160° C., in particular from 90 to 200° C. or more.

According to a further, preferred embodiment, the reactor can have on its under-side an outlet which is connected to a delivery pump by fluid-conducting means. In principle, all pumps known to the person skilled in the art which this person considers suitable for carrying out the process according to the invention, taking into account the properties of the liquid to be delivered, which is optionally also in the form of a suspension, dispersion or emulsion, are suitable as the delivery pump. Preferably, a centrifugal, reciprocating, screw, impeller or hose pump can be employed. A centrifugal pump is very particularly preferred.

According to a further preferred embodiment, a delivery line from the delivery pump is connected to an external heat exchanger by fluid-conducting means, the external heat exchanger being connected to the reactor, preferably to the upper side thereof, by fluid-conducting means. The external heat exchanger is connected to the reactor preferably via a return line of not more than 300 cm to 1 cm length, particularly preferably less than 200 cm to 10 cm length, most preferably less than 100 cm to 40 cm length. Particularly preferably, the external heat exchanger is connected directly, preferably via a flange, to the upper side of the reactor. A plate or tube bundle heat exchanger or a falling film evaporator, or a combination of two or more of these, can be employed as the heat exchanger. A falling film evaporator is preferred.

By the use of an external heat exchanger, the introduction of energy into a delivery stream led via this can be established better both with respect to the duration of the introduction and with respect to the amount of energy, i.e. the heat supplied or removed. This form of introduction of energy renders possible a short duration of the introduction, and therefore little or no change at all to the substance treated in the heat exchanger, in the case of heat-sensitive substances, that is to say those which readily decompose or change. Furthermore, with the use of an external heat exchanger an advantageous ratio of heat transfer area in the heat transfer zone of the heat exchanger to reactor volume can be established.

The delivery stream is preferably led over the heat transfer surface as a film. In this case, the delivery stream has a low height above the heat transfer surface. This arrangement renders possible both a high and a uniform energy transfer rate, so that short energy introduction times are possible compared with other heat transfer arrangements or heat transfer devices. The exposure of the process components to heat in the delivery stream is therefore reduced. Furthermore, undesirable side reactions, e.g. oxidation or polymerization, can likewise be reduced or even avoided.

By suitable choice of the dimensions of the heat transfer surface, in particular of the zone in the flow direction swept over by the film, the volume throughput of the delivery stream and the amount of energy introduced into this can be adapted to the circulation throughput, and thus to the requirements of the process according to the invention. Preferably, a high ratio of heat transfer area in the heat exchanger to volume throughput of the delivery stream is chosen. Furthermore, a ratio of heat transfer area to volume throughput of the delivery stream in a range of from 15 to 1 h/m, particularly preferably from 5 to 1.1 h/m, further preferably from 2 to 1.3 h/m and most preferably from 1.7 to 1.4 h/m is preferred.

For example, the external heat exchanger can be configured as a falling film evaporator. In this case, on entry into the heat exchanger the delivery stream is divided and applied as a film to the inner surfaces of tubes connected to the falling film evaporator inlet by fluid-conducting means and preferably arranged side by side, the tube walls forming the heat transfer surfaces. The sum of the individual heat transfer surfaces of the individual tubes forms the heat transfer surface of the falling film evaporator. On passage of the divided delivery stream from the tube into the outflow of the falling film evaporator, the delivery stream is combined again.

The amount of energy per unit volume of the delivery stream which can be transferred in the heat exchanger is determined by the speed of the delivery stream, the distance flowed over in the direction of flow on the heat transfer surface and by the average thickness of the film when sweeping over the heat transfer surface. The thickness of the film means the height of the film over the heat exchanger surface. Preferably, the thickness of the film is in a range of from 2 to 20%, particularly preferably from 5 to 15%, and furthermore from 7 to 12%, in each case based on the internal diameter of the heat transfer surface constructed as tubes.

A further outlet can be positioned on the reactor under-side. The ester A can be removed from the reactor via this, e.g. by a second delivery pump, after the reaction has ended or been discontinued, and fed to a further processing stage, e.g. a filling unit, a heat exchanger, a processing and/or working up unit. Preferably, in the context of that said so far, the under-side of the reactor has an outlet via which both the delivery stream during the reaction and the ester A are led out of the reactor. In order both to be able to lead the process components over an external heat exchanger as a delivery stream during the reaction, and to be able to lead the ester A via the same outlet after the reaction in the reactor, a delivery pump on the outlet of which a distributing device is arranged is preferably provided on the outlet of the reactor. Several connections leading away are provided on this distributing device, at least a first connection being connected by fluid-conducting means to the external heat exchanger and a second connection to a feed to a further processing stage.

A working up unit is provided as a further processing stage. This has at least one working up container and optionally further devices. All embodiments which are known to the person skilled in the art and appear to be suitable are possible as the working up container. Preferably, the working up container has a tank with a stirrer and heating jacket, it being possible for the heating jacket to be arranged on the inside or outside.

All stirrer types which are known to the person skilled in the art and appear to be suitable for carrying out the process according to the invention can be employed as stirrers. Preferably, stirrer types which have the effect at least in part of axial mixing during operation can be employed. The stirrers can have one or more stirrer tiers, preferably one, 2, 3, 4, 5, 6 or 7 tiers. With respect to the geometry of the stirrer, propeller stirrers are preferred.

The working up unit furthermore has at least one feed, preferably on the upper side of the tank, which is connected to the reactor by fluid-conducting means. The tank furthermore has an outlet, preferably on its under-side. According to a preferred embodiment, a separating device is arranged in a fluid-carrying connection via a delivery pump. A liquid phase separated off in this separating device can be collected and led via a distributing device optionally via a return line into the tank of the working up unit in a circulation, or to a holding unit.

In principle all embodiments which are known to the person skilled and appear to be suitable for carrying out the process according to the invention can be employed as the separating device. For example, filters, centrifuges, separators or filter presses can be employed as the separating device. A filter press is preferably employed. A filter press often has, in a fluid-carrying arrangement, an inlet, at least two filter chambers and at least one recipient with an outlet. Preferably, the filter press has three or more filter chambers, preferably 4, 5, 6, 7, 8, 9, 10, 11, 12, 13, 14, 15, 16, 17, 18, 19, 20, 21, 22, 23, 24, 25, 26, 27, 28, 29 or 30 filter chambers, or a multiple thereof.

Preferably, at least two of the filter chambers are provided with at least one filter frame each. Advantageously, each filter frame is equipped with at least two layers, preferably at least three layer, or more than three layers. For example, a filter frame has a filter material as the first layer, a filter surface as the second layer, and optionally a filter cake as the third layer.

In principle all embodiments which are known and appear to be suitable to the person skilled in the art are possible as filter materials. Filter materials which have a permeability to air in a range of from 5 to 20 l·m⁻²·s⁻¹, preferably from 10 to 20 l·m⁻²·s⁻¹, or from 10 to 15 l·m⁻²·s⁻¹ are preferred according to the invention. The permeability to air can be determined in accordance with DIN 53887. Filter materials of which the weight per unit area is in a range of from 500 to 700 g/m² are furthermore preferred according to the invention. Filter materials which have both the preferred permeability to air, as described above, and the weight per unit area which is preferred according to the invention are furthermore preferred. Porous substances, paper, glass frits, porcelain frits, braids of metal wire, woven fabric or laid fabric of textile or plastics materials are preferably suitable as the filter material. Plastics materials which can be employed are e.g. knitted fabric of plastic based on polyamide, PET, PP, ETFE, PEEK, PVC, or a combination of two or more of these. A multifilament of PVC, polyamide 6, PP and PET is preferred as the filter material.

According to a further preferred embodiment, a filter surface, preferably a flexible filter surface, is arranged on at least one filter material, the filter surface being characterized by at least one of the following features:
FP1) a weight per unit area of 65-75 g/m², particularly preferably 68-72 g/m²;
FP2) a filtration speed of 20″-30″ according to DIN 53137,
FP3) a thickness of 24-30 mm,
FP4) a bursting pressure of 2.5-3.5 kp.

Furthermore, in embodiments according to the invention the filter surface has two or more of the above features. The following combinations of features represented with the aid of the combinations of figures thus result specifically as embodiments: FP1FP2, FP1FP3, FP1FP4, FP2FP3, FP2FP4, FP3FP4, FP1FP2FP3, FP1FP2FP4, FP1FP3FP4, FP2FP3FP4.

According to a further preferred embodiment, the filter surface is formed from, preferably bleached, cellulose. A further preferred embodiment of the filter surface has a combination of polyester fibres and cellulose nonwoven material.

The working up unit furthermore comprises a filter cake, preferably in the separating device. This filter cake is present at least towards the end of the working up, and has a height of between 2 and 10 mm, preferably between 3 and 7 mm. The height here means the thickness of the filter cake perpendicular to the filter surface in the state loaded with liquid. If several filter cakes are present, e.g. if several filter chambers with filter surfaces are arranged side by side, the height of the several filter cakes is to be regarded as the arithmetic mean of the height of the individual filter cakes. In this case, the variation in the height when several individual filter cakes are considered is preferably less than 10%.

According to a further preferred embodiment, a sorbent is present in the working up container. Suitable sorbents and preferred embodiments correspond to those described above.

The reactor can furthermore have at least one reactant reservoir. Any desired installations in which process components can be kept ready before the reaction are conceivable as the reactant reservoir. A storage container, a tank, a boiler or a still is preferred. It is likewise possible to provide a storage container connected to a further production plant as the reactant reservoir.

According to a further, preferred embodiment, the reactant reservoir is connected to the reactor via a line which is led via a preheating stage. All devices known to the person skilled in the art which this person considers suitable for achieving this aim in carrying out the process according to the invention can be employed as the preheating stage. A plate or tube bundle heat exchanger, or a combination of two or more of these, is particularly suitable as the preheating stage. A tube bundle heat exchanger is preferred.

According to a further, preferred embodiment, the reactant reservoir is temperature-controllable, for example by a heating jacket or cooling jacket for the reactant reservoir. It may be desirable here for a substance which is solid at the ambient temperature to be kept ready above its melting point. If this substance is present in liquid form, simple, often also more accurate metering than e.g. in the case of metering of the solid is possible. Furthermore, by leading substances in closed lines, the risk of exposure and contamination of employees and the environment is avoided.

Preferably, the reactor is furthermore connected to a pressure reducing unit. This is preferably arranged in a fluid-carrying continuation of the heat exchanger or heat exchangers and is connected to the end of the line for removal and/or treatment of the fluid stream. In principle all units for generating a reduced pressure which are known to the person skilled in the art are suitable as the pressure reducing unit, as long as he would consider them taking into account the reactor design.

An embodiment, which also contains optional features and is in no way intended to represent a limitation of that said so far is explained further by way of example in the following with the aid of drawings.

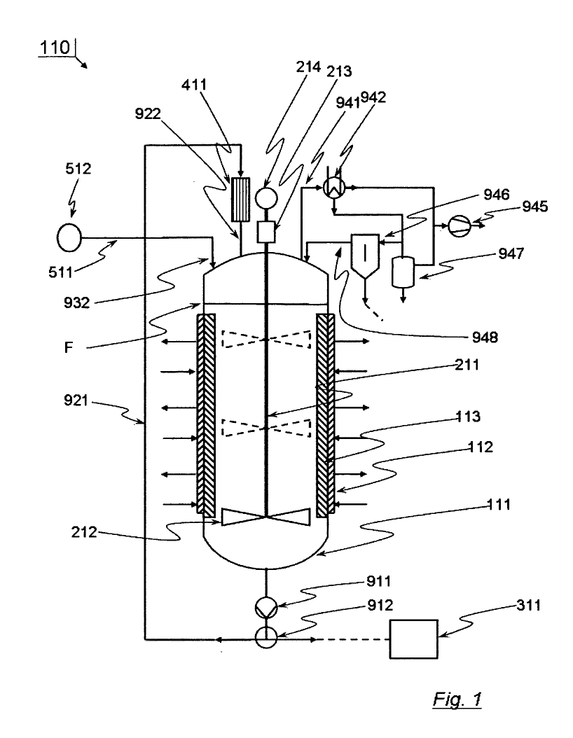

FIG. 1 shows a reaction region 110 with a reactor 111 with various installations which is suitable and preferred for carrying out the process according to the invention. The reactor 111 has on the reactor wall an external heating jacket 112. This is divided into three sections, which can be controlled separately. In the middle of the reactor, along its vertical axis, a stirrer 211 with stirrer tiers 212 is arranged. The stirrer 211 is driven via a transmission 213 with a motor 214. Baffles 113 can be arranged on the reactor wall. On the upper side of the reactor 111, an external heat exchanger 411 is arranged via a connection 922, which can be configured as a return line or as a flange. Preferably, the external heat exchanger 411 is configured as a falling film evaporator. On the under-side of the reactor 111 is an outlet with a shut-off valve, which is connected to a delivery pump 911. A distributing device 912, e.g. a multi-way valve, is attached to the outlet of the delivery pump. From the distributing device 912, a return line 921 leads to the external heat exchanger 411. A second line leads from the distributing device 912 to a working up unit 311. The filling level line F represents the position of the interface between the space underneath the interface taken up by the filling volume and the gas space above this.

On the upper side of the reactor 111, a feed line 511 is attached, which is connected to one or more reactant reservoirs 512 containing process components. Furthermore, a line 941 for removing a fluid stream leads from the upper side of the reactor 111 to the heat exchanger 942. This is connected to a second heat exchanger 943. One or more cooling fluids flow through the heat exchangers 942 and 943 at the same or a different temperature. The exits of the heat exchangers 942 and 943 are connected to a receiver 947 and a separator 946. A condensate of the heat exchangers 942 and 943 can be fed either directly or via the receiver 947 and a further heat exchanger 944 to the separator 946. The separator 946 has an interfacial layer regulator, from which a return line 948 leads to the reactor 111. The separator 946 and the receiver 947 can likewise be emptied by an outlet in each case arranged on the under-side. A reduced pressure can be generated by a pressure reducing unit 945 via a line connected to the heat exchanger 944 and the receiver 947.

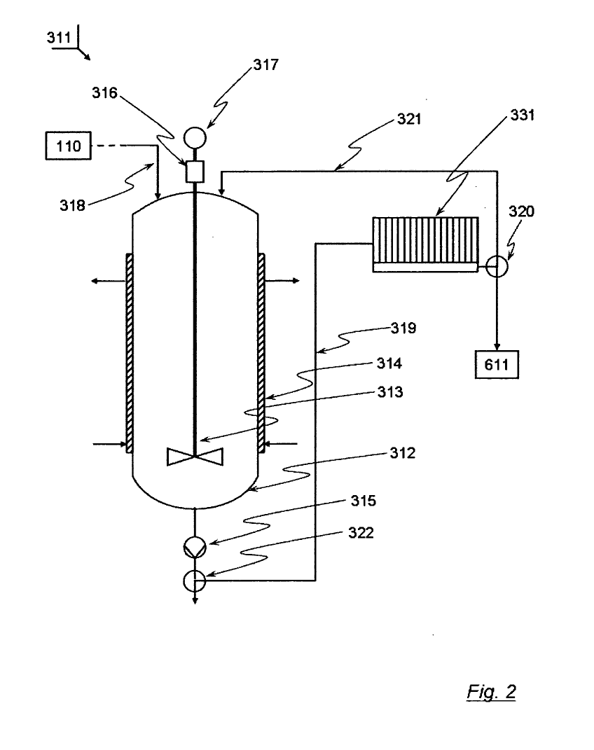

The working up unit 311, with a working up container 312 and a filter press 331, is shown by way of example in FIG. 2: From the reactor 111, a feed 318 which leads on the upper side of the working up container 312 into this is attached. The working up container 312 has a mixing device 313 driven by a motor 317 via a transmission 316. A heating jacket 314, externally here, is provided on the wall of the working up container. From the under-side of the working up container 312, a delivery line 319 leads via a delivery pump 315 to a filter press 331, the outlet of which is provided with a distributing device 320. From the distributing device 320, a return line 321 leads to the upper side of the working up container 312 and a further line leads to a holding unit 611.

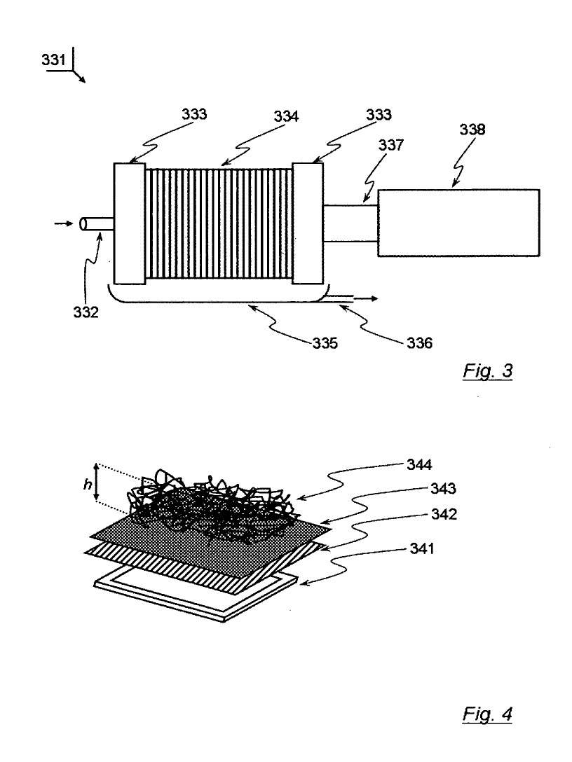

The filter press 331 has the following components (FIG. 3): A feed line 332 is connected to a first front plate 333. Filter chambers 334 which are held together by a ram 337 driven by a motor with transmission 338 are arranged between the first front plate 333 and a further front plate 333. Under the filter press 221, at least under the filter chambers, a recipient 335 with an outlet 336 is arranged.

According to FIG. 4, each filter chamber 334 comprises a filter frame 341, on which are positioned a filter material 342 and a filter surface 343. Furthermore, a filter cake 344 of thickness (height) h can be positioned on the filter surface 343.

The present invention also provides a process for the preparation of a formulation comprising the components
a1) a base liquid, and
b1) at least one ester, the ester being obtainable by the process according to the invention, and
c1) optionally further additives,
   comprising the process steps:
i) provision of the base liquid,
ii) provision of the at least one ester,
iii) optionally provision of further additives,
iv) mixing of components i), ii) and optionally iii).

All liquids which are known to the person skilled in the art and appear to be suitable are possible as base liquids which can be employed according to the invention. Both aqueous and non-aqueous liquids, e.g. water, hydrocarbons, oils or other organic substances which are liquid at 20° C., are particularly suitable.

According to a preferred embodiment, the base liquid is an oil. In this connection, oil is understood as meaning organic compounds which preferably form with water in a ratio of from 5:95 to 95:5 a separate oil phase which comprises more than 90%, preferably more than 95%, most preferably more than 99% of the total amount of oil.

Substances chosen from the group of
αα1) esters from monofunctional saturated or unsaturated, linear or branched alcohols having 1 to 24 carbon atoms and monofunctional saturated or unsaturated, linear or branched fatty acids having 1 to 24 carbon atoms, and mono- and polyfunctional, linear or branched alcohols having 6 to 36 carbon atoms;
αα2) mineral oils, diesel oils, paraffin oils;
αα3) linear α-olefins and derivatives thereof and internal olefins;
αα4) carbonic acid esters; or
αα5) a mixture of two or more of the substances described in αα1) to αα4),
are possible in particular as the oil.

Suitable esters under the term “ester oils” are described in the European patents of the applicant EP-A-0374671, EP-A-0374672, EP-A-0386638, EP-A-0386636 and EP-A-0535074. The disclosure thereof is a part of the present invention.

Certain water-insoluble alcohols are furthermore suitable as the base liquid, water-insoluble meaning, as above, a water-solubility of less than 100 g of oil/l of water. Polyfunctional, in particular di- or trifunctional water-insoluble alcohols are preferably employed.

Linear α-olefins and derivatives thereof, in particular poly-α-olefins (PAO), are furthermore suitable as the base liquid. Suitable compounds of this type are described e.g. in the international laid-open specification WO-A-95/34610. Furthermore, internal olefins are preferred as the base liquid.

Carbonic acid esters such as are described e.g. in EP-A-0532570 are furthermore employed as the base liquid. It is furthermore possible in principle, and particularly preferable, to combine to a mixture, which is also to be called base oil here, and employ several of the abovementioned substances, in particular also from several of the abovementioned groups of base oils. The base liquids described above are preferred in particular if the formulation according to the invention is a drilling mud.

Further additives can furthermore be employed as process component c1). In the case of drilling fluids, these are, inter alia, weighting agents, fluid loss additives, alkali reserves, viscosity regulators or the like, or two or more of these, which are the subject matter of extensive general literature and relevant patent literature.

If the formulation is a drilling mud and the base liquid is an oil, in general invert emulsion slurries which consist of a three-phase system: oil, water and finely divided solids, are employed. These are formulations of the W/O emulsion type, that is to say the aqueous phase is distributed in the continuous oil phase in a heterogeneous, finely disperse manner.

According to a preferred embodiment, the drilling mud has a content of

10 to 50 wt % of an organic base liquid, and
0.3 to 5 wt. % of ester, and
50 to 85 wt. % of further additives,
the sum of all the wt. % being 100.

According to a further preferred embodiment of the process according to the invention for the preparation of a formulation, the formulation is a metal working liquid, which can be aqueous or have a low water content. Low water content means that the formulation contains less than 10 wt. %, based on the total formulation, of water. Metal working liquids are used as lubricants and extreme pressure additives in working processes such as e.g. cutting, drilling, milling, thread cutting, turning, ejecting or grinding. In such processes, the tool and the workpiece are conventionally flushed with liquid in order to remove the heat generated.

In the case of a metal working liquid of low water content, the process preferably comprises

40 to 99.6 wt. % of a base liquid which is liquid at 20° C. or a mixture of two or more of these,
0.4 to 10 wt. % of a wax or a mixture of several waxes,
0.5 to 10 wt. %, particularly preferably 1 to 6 wt. % and most preferably 2 to 4 wt. % of the ester according to the invention, and
1 to 25 wt. % of additives,
the sum of the constituents being 100 wt. %. Such a metal processing liquid of low water content is described e.g. in DE-A-10115696.

In connection with metal working liquids, particularly preferred possible base liquids are those liquids of which the viscosity is higher than that of water under the same conditions and which do not mix with water according to the description given above. Examples of these are mineral oils based on paraffins or naphthene or ester oils on a natural (i.e. plant or animal) or synthetic basis. Mineral oils are suitable in particular. Preferably, these oils have at 20° C. a viscosity, measured in accordance with DIN 53211, in the range of from 2 to 500 mm²/sec. Preferably, the metal processing liquid contains 55 wt. % or more, in particular 60 wt. % or more and most preferably 70 wt. % or more of base liquid.

All natural waxes, modified waxes or synthetic waxes which are known to the person skilled in the art and appear to be suitable can be employed as waxes. Montan wax, carnauba wax or polyethylene wax is particularly suitable.

Preferably, a metal processing liquid contains 0.4 wt. % or more, in particular 0.6 wt. % or more of wax. The upper limit of wax is preferably 5 wt. %, particularly preferably 3 wt. %. It may be particularly favourable to combine different types of wax, e.g. montan wax with polyethylene wax.

Preferred lubricating additives which can be employed in addition to the ester or ester mixture according to the invention are, in particular, high performance lubricating additives (so-called “EP additives”) from the English expression “extreme pressure additive”, which are preferably chosen from sulphur- or phosphorus-containing EP additives. Sulphur-containing fatty acid esters, dialkyl trisulphides, dialkylene pentasulphides, e.g. di-t-dodecyl polysulphite, and neutralized phosphoric acid esters are particularly preferred.

In the case of an aqueous metal working liquid, this is preferably an oil-in-water emulsion, in which the ester according to the invention is present in the form of finely dispersed droplets in an aqueous phase. Such aqueous metal working liquids are described, for example, in WO-A-91/15455. Preferably, an emulsion concentrate in the form of a water-in-oil emulsion or an oil-in-water emulsion, which is then diluted accordingly with water before its use, is provided for the preparation of an aqueous metal working liquid.

According to a further preferred embodiment of the formulation according to the invention or of the process according to the invention for the preparation of a formulation, the formulation is a hydraulic oil. Hydraulic oils are liquids which act by transmitting pressures to hydraulic drives or hydraulic control and regulating equipment. Since the wear on the regulating organs is to be kept as low as possible, hydraulic oils also always have lubricating properties.

The hydraulic oils according to the invention preferably comprise a base oil, the fatty acid ester mixture according to the invention and optionally further additives. In this context, those base oils which are mentioned as preferred “base oils” in DE-A-43 13 948 are preferred in particular as base oils. Additives which can also be added, in addition to the dicarboxylic acid esters, described in DE-A-43 13 948, of Guerbet alcohols, are oxidation inhibitors, such as derivatives of sulphur, phosphorus, phenol, or also amines, detergents, such as naphthenates, stearates, sulphonates, phenolates, phosphates, or also EP additives, such as compounds of sulphur and chlorine, foam prevention agents, demulsifiers, corrosion inhibitors or agents which lower the coefficient of friction. The addition of so-called viscosity index improvers is also possible. Such additives can be added in conventional amounts.

The hydraulic oil according to the invention preferably contains the ester according to the invention in amounts of from 0.5 to 10 wt. %, particularly preferably from 1 to 6 wt. % and most preferably 2 to 4 wt. %, in each case based on the total hydraulic oil.

According to the invention, the base liquid, the at least one ester, and optionally further additives are provided and then mixed. The mixing can be carried out in any manner in principle which is known to the person skilled in the art and appears to be suitable. Mixing with a high-speed propeller or with a static mixer is preferred in particular. The sequence in which the various components are brought together is of minor importance here.

According to a further preferred embodiment of the process according to the invention for the preparation of a formulation, the at least one ester, preferably all of the esters employed, has a pour point determined in accordance with the test method described herein of a maximum of −10° C., preferably a maximum of −15° C., or a maximum of −20° C., determined in accordance with DIN ISO 3016.

The present invention also provides a formulation which is obtainable by the process according to the invention described above.

According to a preferred embodiment, the formulation according to the invention is chosen from the group consisting of:

a drilling mud, a metal working liquid or a hydraulic liquid.

The present invention also provides a further processing product comprising
a) an ester obtainable by the process according to the invention described above, as an additive, and
b) at least one functional component chosen from the group consisting of thermoplastic polymer, enzyme, curing agent of an adhesive, paraffin, oil, colouring agent, hair or skin care substance, polymer dispersion, lime mud, lubricant or emulsifier, or a combination of two or more of these.

The present invention also provides the use of an ester obtainable by the process according to the invention described above as an additive in a composition, the composition being chosen from the group consisting of: a thermoplastic composition, a detergent, an adhesive, a defoamer, a lubricant formulation, a lacquer, a paint, a cosmetic formulation, a soil compacting agent, a drilling mud, a hydraulic oil or a dispersion.

According to a further preferred embodiment, the ester is used as an additive in a composition comprising as a functional component
α) a thermoplastic polymer, the composition being a thermoplastic composition;
β) an enzyme, the composition being a detergent;
γ) a curing agent of an adhesive, the composition being an adhesive;
δ) a paraffin, the composition being a defoamer;
ε) an oil, the composition being a lubricant formulation;
ζ) a colouring agent, the composition being a lacquer or a paint; or
η) a hair or skin care substance, the composition being a cosmetic formulation,
θ) a polymer dispersion, the composition being a soil compacting agent,
ι) a lime mud, the composition being a drilling mud,
κ) a lubricant, the composition being a hydraulic oil,
λ) an emulsifier, the composition being a thermoplastic composition or a dispersion,
   wherein the carboxylic acid component comprises at least 50 wt. % of at least one mono- or polyunsaturated, aliphatic carboxylic acid, based on the total weight of all the carboxylic acid components,
   wherein the alcohol component contains at least one polyol which is solid at 25° C.,
   wherein the ester has preferably been obtained by the process according to the invention described above for the preparation of an ester comprising process steps i., ii. and optionally iii.

Preferably, the additive is employed in an amount in a range of from 0.001 to 40 wt. %, particularly preferably in a range of from 0.01 to 20, very particularly preferably from 0.1 to 10 wt. % and particularly preferably in a range of from 0.5, 1 or 2 to 5, 6, 7 or 8 wt. %, based on the composition.

The invention is now explained in more detail with the aid of non-limiting examples.

### Measurement Methods

Unless expressly stated otherwise, all the measurements are carried out in accordance with the relevant ISO standards. Unless specified otherwise there, a temperature of 23° C., an atmospheric pressure of 1 bar and a relative atmospheric humidity of 50% was chosen.

### Composition of a Mixture of Several Carboxylic Acid Components

Mixtures of several carboxylic acid components such as are present, for example, in technical grade oleic acid can be determined by means of gas chromatography (GC) or high pressure liquid chromatography (HPLC). The weight contents are stated in wt. %, based on the total weight of the sample supplied.

### Further Methods

The following characteristic values are determined in accordance with published standards:

| Characteristic value | Standard | Comments |
|---|---|---|
| BET surface area | DIN 66131 | with nitrogen |
| Hydroxyl number (OHN) | DIN 53240 | |
| Acid number (AN) | DIN EN ISO 3682 | |
| Saponification number (SN) | DIN 3657 | |
| Iodine number | EN ISO 3961 | |
| Chain distribution | ISO 5508 | |
| Pour point | DIN ISO 3016 | |
| Cloud point | DGF D-III 3 | |
| Glass transition temperature | DIN 53765 | see above |
| (T_{g}), melting point (Tₘ) | | |
| Density | DIN 51757 | at 20° C. |
| Viscosity | DIN 53015 | at 20° C. |
| Gardner colour number | DIN EN ISO 4630-1 | |
| Lovibond colour number | BSI BS 684 | |
| Hazen colour number | DIN ISO 6271 | |
| Thickness of the multifilament | DIN 53855 Part 1 | |
| [μm] | | |
| Particle size by means of laser | ISO13320-1 | with Coulter 230 LS |
| diffractometry | | |
| Water content | DIN 51777 | |

### EXAMPLES

Unless noted otherwise, the raw materials, obtainable under the trade names given, are obtainable from Cognis Oleochemicals Deutschland GmbH, Düsseldorf, or from Sigma-Aldrich Chemie GmbH, Steinheim.

### Example 1

### Preparation of Pentaerythritol Dioleate

621.4 g of technical grade oleic acid (2.2 mol) and 136 g (1 mol) of pentaerythritol were initially introduced into a glass flask and 0.2 g of tin oxalate was added. The mixture was heated from 130 to 180° C. in the course of 3 hours and at 210° C. for a further 4 hours. After the mixture had been cooled, this was washed eight times with 10 g of water and the pentaerythritol dioleate obtained in this way was dried in vacuo (p approx. 16 mbar) at 95-100° C. for 3 hours. Finally, 30 g of kieselguhr was stirred into the pentaerythritol dioleate and the suspension obtained in this way was filtered into a suction flask. The filtrate was dried again at 100° C. in vacuo (p approx. 16 mbar) over a period of one hour.

A yellowish liquid was obtained; the yield was 87%, based on the pentaerythritol weighed out. The acid number was 1.2.

### Example 2

### Preparation of a Thermoplastic Composition

6 kg of polyethylene terephthalate (PET SP04 from Catalana de Polimers) was introduced into a 15 kg Henschel mixer. The mixing wall temperature was 40° C. 0.5 wt. % of the ester prepared in Example 1 was furthermore added as a mould release agent. The material was then granulated on a granulator (ZSK 26Mcc) with a stuffing screw.

### Example 3

### Production of a Shaped Article

For production of shaped articles from the thermoplastic composition prepared in Example 2, a fully hydraulic injection moulding machine with a hydraulic closing unit of the Battenfeld HM800/210 type was employed. The maximum closing force is 800 kN, the screw diameter is 25 mm. A mould with a conically tapering, rectangular core was used at the test mould. For determination of the demoulding force, a load cell with a maximum measuring range of 2 kN was attached to the ejector rod. The moulding composition was predried at about 225° C. for about 4 hours. Significantly improved demoulding was observed with the thermoplastic composition according to the invention compared with an additive-free moulding composition.

### Example 4

### Preparation of a Lubricant

For the preparation of an oil-based drilling mud with a lubricant, the following constituents were brought together: mineral oil (675 ml), water (225 ml), calcium chloride (95 g), emulsifier (35 g), fluid loss additive (10 g), viscosity-forming agent (25 g), lime (17 g), barite (360 g), and 1.5 wt. %, based on the total weight of all the abovementioned constituents, of pentaerythritol dioleate from Example 1. In comparison with a mud without pentaerythritol dioleate, the mud according to this example has the effect of lower friction. Furthermore, practically no foaming was observed.

### Example 5

### Preparation of a Soil Compacting Agent

To a commercially available polyvinyl acetate dispersion (approx. 50 wt. % of polyvinyl acetate, obtainable from Henkel KGaA, Düsseldorf), 10 wt. %, based on the aqueous dispersion, of
a) pentaerythritol dioleate from Example 1,
b) oleic acid n-octyl ester, or
c) sebacic acid di(n-butyl ester) is added.

As experiment d), a film was obtained from the untreated dispersion, which serves as a reference. Films with an average thickness of (400±30) μm are produced from these dispersions a) to d) by dipcoating on rotating Teflon discs of 10 cm diameter.

These films are evaluated with respect to flexibility, water-solubility and cohesion. After 21 days, they were tested for stability and homogeneity.

In comparison with the reference experiment d), all three dispersions a) to c) show a significantly improved flexibility of the films after three weeks. Films a) to c) moreover show an extremely high cohesion compared with d) immediately after film formation and in particular also after three weeks, so that they tear only when a high force is applied.

Field experiments in which aa) layers of sand, bb) potting compost and cc) sandy loess were treated with dispersions a) to d) and exposed to weathering in the Düsseldorf, Germany region for 3 weeks proved to be highly compacted in the case of a) to c) compared with the reference d).

### LIST OF REFERENCE SYMBOLS ON FIG. 1

| | |
|---|---|
| Reaction region | 110 |
| Reactor | 111 |
| Heating jacket | 112 |
| Baffle | 113 |
| Stirrer shaft | 211 |
| Stirrer blade | 212 |
| Transmission | 213 |
| Motor | 214 |
| Working up unit | 311 |
| External heat exchanger | 411 |
| Feed line | 511 |
| Reactant reservoir | 512 |
| Delivery pump | 911 |
| Distributing device | 912 |
| Circulation line | 921 |
| Return line, flange | 922 |
| Feed of process components | 932 |
| Vapours line | 941 |
| Heat exchanger | 942 |
| Pressure reducing unit | 945 |
| Separator with interfacial layer | 946 |
| Receiver | 947 |
| Return line | 948 |
| Filling level line | F |

### LIST OF REFERENCE SYMBOLS ON FIG. 2 (OBJECT 311)

| | |
|---|---|
| Reaction region | 110 |
| Working up unit | 311 |
| Working up container | 312 |
| Mixing device | 313 |
| Heating jacket | 314 |
| Delivery pump | 315 |
| Transmission | 316 |
| Motor | 317 |
| Feed | 318 |
| Delivery line | 319 |
| Distributing device | 320 |
| Return line | 321 |
| Distributing device | 322 |
| Filter press | 331 |
| Holding unit | 611 |

### LIST OF REFERENCE SYMBOLS ON FIG. 3 (OBJECT 331)

| | |
|---|---|
| Filter press | 331 |
| Feed | 332 |
| Front plate | 333 |
| Filter chamber | 334 |
| Recipient | 335 |
| Outlet | 336 |
| Ram | 337 |
| Motor with transmission | 338 |

### LIST OF REFERENCE SYMBOLS ON FIG. 4 (OBJECT 334)

| | |
|---|---|
| Filter frame | 341 |
| Filter material | 342 |
| Filter surface | 343 |
| Filter cake | 344 |

1. A process for the preparation of an ester, at least based on
a. at least one alcohol component,
b. at least one carboxylic acid component,
c. optionally further additives, and
d. at least one catalyst
as process components, comprising, in a reactor, the process steps:
i. provision of the process components,
ii. reaction of the process components to give an ester A,
iii. after-treatment of the ester A,
wherein
the alcohol component comprises at least one polyol which is solid at 25° C., and
the carboxylic acid component comprises at least 50 wt. % of at least one mono- or polyunsaturated, aliphatic carboxylic acid, based on the total weight of the carboxylic acid components, and
wherein a pressure in a range of 2-600 mbar is applied to the reactor at least during a part of the reaction.

2. The process according to claim 1, wherein the after-treatment of the ester A comprises the following steps:
aa. provision of the ester A,
bb. addition of water in an amount of from 1 to 10 wt. %, based on the weight of the ester A,
cc. mixing to give an aqueous phase,
dd. separating off of the aqueous phase to give an ester B,
ee. optionally drying of the ester B,
ff. optionally treatment with a sorbent,
the after-treatment being carried out at a temperature of 70-100° C.

3. The process according to claim 2, wherein steps aa. to dd. are repeated twice to ten times, during the second and each further time in each case the purified ester B from step dd. of the preceding time being provided as ester A in step aa.

4. The process according to claim 2 or 3, wherein the ester B is dried in step ee. at a temperature of 90-150° C., preferably under a pressure of from 2 to 600 mbar.

5. The process according to one of claims 2 to 4, wherein the ester B is combined in step ff. with a sorbent to give a mixture, before this mixture is divided into a solid and a liquid phase, the ester B being obtained as the liquid phase.

6. The process according claim 5, wherein the sorbent has a BET surface area in a range of from 0.5 to 7 m²/g.

7. The process according to one of claim 5 or 6, wherein the sorbent has at least one of the following features:
a particle size distribution with a maximum between 15 and 75 μm;
an average particle size in a range of from 20 to 40 μm;
a content in a range of from 40 to 60 wt. %, based on the total amount of sorbent, with a particle size in a range of from 16 to 72 μm;
an average pore size in a range of from 5 to 10 μm.

8. The process according to one of claims 5 to 7, wherein silica gel, kieselguhr, active charcoal, bentonite, montmorillonite or zeolite, in particular kieselguhr, is employed as the sorbent.

9. The process according to one of the preceding claims, wherein
pentaerythritol, pentaerythritol dimer or a pentaerythritol oligomer is chosen as the alcohol component,
a carboxylic acid mixture comprising at least 50 wt. % of oleic acid is chosen as the carboxylic acid component,
a molar ratio of carboxylic acid groups of the carboxylic acid component to alcohol groups of the alcohol component of from 0.2 to 0.8 being established.

10. The process according to one of the preceding claims, wherein a ratio of unsaturated C₁₆-carboxylic acids to unsaturated C₁₈-carboxylic acids of from 1:5 to 1:20 is present in the carboxylic acid component.

11. The process according to one of the preceding claims, wherein the carboxylic acid component comprises an amount of less than 25 wt. %, preferably less than 20 wt. %, or less than 13 wt. %, of saturated carboxylic acids.

12. The process according to one of the preceding claims, wherein the catalyst is employed in an amount of 0.01-5.0 wt. %, based on the total weight of the sum of the alcohol components and carboxylic acid components.

13. The process according to one of the preceding claims, wherein the carboxylic acid component has been obtained from beef tallow.

14. The process according to one of the preceding claims, wherein a catalyst which comprises one or more compounds chosen from the group consisting of divalent tin compounds, p-toluenesulphonic acid, methanesulphonic acid, sulphuric acid, hypophosphorous acid, in particular tin oxalate, tin oxide, tin octoate, is employed as an additive.

15. The process according to claim 14, wherein 0.01 to 0.08 wt. %, based on the total amount of process components a. and b., of tin oxalate is employed as the catalyst.

16. The process according to one of the preceding claims, wherein the reaction is carried out at a temperature in a range of from 150 to 250° C.

17. The process according to one of the preceding claims, wherein the ester has at least one of the following features:
a Gardner colour number of 7 or less,
a water content of less than 0.1 wt. %, based on the total weight of the ester,
an acid number of less than 2,
a hydroxyl group number (OHN) in a range between 90 and 200,
a melting point in a range of from −50 to −20° C.,
a cloud point in a range of from −20 to −5° C.

18. A device comprising as device units connected by fluid-conducting means
α) at least one reactant reservoir (512),
β) a reactor (111) with a mixing device (211, 212),
γ) a working up unit (311),
wherein the working up unit (311) comprises, connected by fluid-conducting means:
αα) a working up container (312),
ββ) a delivery pump (315) and
γγ) a separating device (331),
wherein a filter press which has 2 or more filter chambers (334) which comprise at least one filter material (342), the filter material (342) having a permeability to air of from 5 to 20·l·m⁻²·s⁻¹ and a weight per unit area of from 500 to 700 g/m², is employed as the separating device (331).

19. The device according to claim 18, wherein the working up container (312) has a head region with a distributing device with nozzles.

20. The device according to claim 18 or 19, wherein a filter surface (343) is arranged on each filter material (342), the filter surface (343) being characterized by at least one of the following features:
FP1) a weight of 65-75 g/m²,
FP2) a filtration speed of 20″-30″ according to DIN 53137,
FP3) a thickness of 24-30 mm,
FP4) a bursting pressure of 2.5-3.5 kp.

21. The device according to one of claims 18 to 20, wherein a filter cake (344) forms in at least one filter chamber (334), this filter cake (344) having a height of between 2 and 10 mm.

22. The device according to one of claims 18 to 21, wherein a sorbent is present in the working up unit (311).

23. The device according claim 22, wherein the sorbent in the working up container has a BET surface area in a range of from 0.5 to 7 m²/g.

24. The device according to one of claim 22 or 23, wherein the sorbent has at least one of the following features:
a particle size distribution with a maximum between 15 and 75 μm;
an average particle size in a range of from 20 to 40 μm;
a content in a range of from 40 to 60 wt. %, based on the total amount of sorbent, with a particle size in a range of from 16 to 72 μm;
an average pore size in a range of from 5 to 10 μm.

25. The device according to one of claims 22 to 24, wherein silica gel, active charcoal, bentonite, montmorillonite or zeolite, in particular kieselguhr, is employed as the sorbent.

26. A process for the preparation of an ester, wherein a device according to one of claims 18 to 25 is employed.

27. A process for the preparation of a formulation comprising the components
a1) a base liquid, and
b1) at least one ester, the ester being obtainable by a process according to one of claims 1 to 17 or claim 26, and
c1) optionally further additives,
comprising the process steps:
i) provision of the base liquid,
ii) provision of the at least one ester,
iii) optionally provision of further additives,
iv) mixing of components i), ii) and optionally iii).

28. The process according to claim 27, wherein the base liquid is an oil.

29. The process according to claim 27 or 28, wherein the at least one ester, preferably all of the esters employed, has a pour point determined in accordance with the test method described herein of a maximum of −10° C., preferably a maximum of −15° C., or a maximum of −20° C., determined in accordance with DIN ISO 3016.

30. A formulation obtainable by a process according to one of claims 27 to 30.

31. The formulation according to claim 30, wherein the formulation is chosen from the group consisting of a drilling mud, a metal working liquid, or a hydraulic liquid.

32. Further processing product comprising an ester which can be prepared by a process according to one of claim 1 to 17 or 26, as an additive, and at least one functional component chosen from the group consisting of thermoplastic polymer, enzyme, curing agent of an adhesive, paraffin, oil, colouring agent, hair or skin care substance, polymer dispersion, lime mud, lubricant or emulsifier, or a combination of two or more of these.

33. Use of an ester obtainable by a process according to one of claim 1 to 17 or 26 as an additive in a composition which is chosen from the group consisting of a thermoplastic composition, a detergent, an adhesive, a defoamer, a lubricant formulation, a lacquer, a paint, a cosmetic formulation, a soil compacting agent, a drilling mud, a hydraulic oil or a dispersion.

34. The use according to claim 33, wherein the additive is employed in an amount in a range of from 0.001 to 40 wt. %, based on the composition.
